(19) United States
(12) Patent Application Publication
(10) Pub. No.: US 2026/0232693 A1
(43) Pub. Date: Aug. 13, 2026

(54) IMPROVING GLYMPHATIC-LYMPHATIC EFFLUX

(71) Applicant: UNIVERSITY OF ROCHESTER, Rochester, NY (US)

(72) Inventors: Maiken Nedergaard, Rochester, NY (US); Ting Du, Rochester, NY (US); Steven A. Goldman, Webster, NY (US)

(73) Assignee: UNIVERSITY OF ROCHESTER, Rochester, NY (US)

(21) Appl. No.: 19/154,705

(22) PCT Filed: Feb. 15, 2024
(86) PCT No.: PCT/US24/15976

§ 371 Date: Aug. 7, 2025

Related U.S. Application Data
(60) Provisional application No. 63/485,278, filed on Feb. 16, 2023.

(51) Int. Cl. A61K 31/5575; A61P 25/00

(52) U.S. Cl. CPC A61K 31/5575; A61P 25/00

(57) ABSTRACT

The present disclosure relates to improving a glymphatic/lymphatic solute efflux from the central nerve system.

### CROSS REFERENCE TO RELATED APPLICATION

This application claims priority to U.S. Provisional Application No. 63/485,278 filed on Feb. 16, 2023. The contents of the application is incorporated herein by reference in its entirety.

### GOVERNMENT INTERESTS

This invention was made with government support under NS100366 and AG057575 awarded by the National Institutes of Health and under W911NF1910280 awarded by the U.S. Army Research Office. The government has certain rights in the invention.

### FIELD OF THE INVENTION

The present disclosure relates to improving a glymphatic/lymphatic solute efflux from the central nerve system.

### BACKGROUND

Lymphatic vessels (LVs) are responsible for returning 4-5 L of fluid from the interstitial space back into venous circulation daily. Failure in their structure or function leads to catastrophic disease such as lymphedema and contributes to the development of cancer metastasis, autoimmune disorders (e.g., inflammatory bowel disease), glaucoma, obesity, and cardiovascular disease. The central nervous system (CNS) lacks LVs, but recent work has shown that a network of astrocyte-connected perivascular spaces (glymphatic system) facilitate transport to LVs in the surrounding meninges^{1,2} and along exiting cranial and spinal nerves³ to drain fluid and solute to the periphery and into venous circulation.

Cervical lymphatic vessels (cLVs) in the neck are responsible for draining ~50% of cerebrospinal fluid (CSF) from the brain and into cervical lymph nodes (cLNs)⁴⁻⁶ and this drainage pathway has shown to play a role in the development of Alzheimer's disease^{7 8} and Parkinson's disease and recovery after stroke and traumatic brain injury. Transport of CNS antigens to the periphery via cLVs also influences the development of multiple sclerosis and modulates the effectiveness of immune checkpoint inhibitors in CNS tumours and amyloid-β immunotherapy. Cervical LVs also contribute to fluid homeostasis and their ligation can increase intracranial pressure⁹ and contribute to cerebral edema¹⁰ suggesting that healthy cLVs are crucial for efficient fluid and solute clearance from the brain. However, despite their importance, the hydrodynamical properties of these vessels have never been directly assessed in vivo and this is of particular importance since their function declines in aging^{7,11,12} and coincides with the increasing risk of lymphatic-related CNS diseases^{5,13,14}. There is a need for improving glymphatic-lymphatic efflux.

### SUMMARY

This disclosure addresses the need mentioned above in a number of aspects.

In one aspect, this disclosure provides a method for improving glymphatic-lymphatic efflux from the CNS of a subject. The method comprises enhancing the flow rate of a cervical lymphatic vessel (cLV) of the subject.

In some embodiments, the enhancing the flow rate comprises increasing the contraction of the cLV. In some embodiments, the increasing the contraction of the cLV comprises administering to the subject an agonist of smooth muscle contraction. In some embodiments, the agonist comprises a muscarinic agonist, an α1-adrenergic agonist, a K_{ATP} channel inhibitor, a NOS inhibitor, a, soluble guanylate cyclase (sGC) blocker, a PKA inhibitor, a cyclooxygenase-1 (COX-1) inhibitor, a cyclooxygenase-2 (COX-2) inhibitor, a prostanoid, a prostaglandin E2 receptor 4 (EP4 receptor) antagonist, a prostacyclin (PGI2) receptor (IP receptor) antagonist, or a nonsteroidal anti-inflammatory drug (NSAID). In some embodiments, the agonist is selected from the group consisting of carbachol, norepinephrine, propranolol, glibenclamide, NG-nitro-L-arginine (L-NNA), ODQ, H-89, KT5720, indomethacin, ketoprofen, AH23848B, GW627368x, CAY10441, pyridostigmine, amifampridine, neostigmine, edrophonium, and ambenonium. In some embodiments, the agonist comprises the prostanoid. In some embodiments, the prostanoid is a lymphatic contractility-promoting prostanoid, such as prostaglandin F_{2α} (PGF_{2α}) thromboxane A₂ (TxA₂), PGD₂, leukotriene B4, leukotriene C4, leukotriene D4, or TxA₂ mimetic U46619.

In some embodiments, improving glymphatic-lymphatic efflux or the flow rate from the CNS of a subject comprises administering to the subject an inhibitor of phosphodiesterase type 5a.

In some embodiments, the agonist or inhibitor is administered to the neck of the subject. In some embodiments, the agonist or inhibitor is administered topically.

In some embodiments, the method further comprises one or more of the following: increasing influx of cerebrospinal fluid (CSF) through the CNS interstitium, and messaging the neck of the subject. In some embodiments, the increasing influx of CSF comprises administering to the subject an agent selected from the group consisting of a hypertonic solution a Stat-3 inhibitor, a bone morphogenetic protein (BMP) signaling axis molecule, an antagonist of AVP (vasopressin), an antagonist of atrial natriuretic peptide (ANP), an antagonist of Angiotensin II, an antagonist of AT2R receptors, and an antagonist of AT1 receptors. In some embodiments, the hypertonic solution comprises NaCl or mannitol.

Another aspect of the disclosure provides a method for promoting clearance of a substance, (e.g., a waste product) from the CNS interstitium, brain interstitium and/or spinal cord interstitium of a subject. The method comprises improving glymphatic-lymphatic efflux from the CNS of the subject in the manner described above. In some embodiments, the substance comprises a fluid or a solute. In some embodiments, the substance comprises amyloid β (Aβ), tau, or alpha synuclein. In some embodiments, the substance comprises a drug or a metabolite thereof. Examples of the drug include small molecule compounds (e.g., chemotherapy compounds), biologics (e.g., proteins, antibodies, nucleic acids, and vectors), and others (e.g., liposome, nanoparticles).

A further aspect of this disclosure provides a method for treating a cerebral edema, a traumatic brain injury, a post-traumatic brain injury, a neurological disorder or a neurodegenerative disease in a subject in need thereof. The method comprises improving glymphatic-lymphatic efflux from the CNS of the subject in the manner described above.

One aspect of this disclosure provides a method for treating a cerebral edema, a traumatic brain injury, a post-traumatic brain injury, or a neurodegenerative disease in a subject in need thereof. The method comprises administering to the subject an inhibitor of phosphodiesterase type 5a. In one embodiment, the inhibitor is selected from the group consisting of sildenafil, tadalafil, vardenafil, and avanafil.

In some embodiments, the neurodegenerative disease is Parkinson's disease (PD), Alzheimer's disease (AD), Alzheimer's disease with Lewy bodies, Lewy body dementia, mixed dementia, vascular dementia, frontotemporal dementia, chronic traumatic encephalopathy (CTE), HIV associated dementia, Lewy body disease, Huntington disease, or multisystem atrophy. In some embodiments, the method further comprises administering to the subject a drug for treating the neurodegenerative disease. In some embodiments, the subject is a mammal. In some embodiments, the mammal is a human. In some embodiments, the mammal or human is an aged mammal or human.

The details of one or more embodiments of the disclosure are set forth in the description below. Other features, objectives, and advantages of the disclosure will be apparent from the description and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A, 1B, 1C, 1D, 1E, 1F, 1G, and 1H diagrams visualizing CSF transport through superficial cervical lymph vessels. a. cLVs in anesthetized mice were surgically exposed, and intravital two-photon microscopy was used to image the flow in the exposed vessel and track the motion of the vessel walls. Heart rate and respiration were simultaneously recorded. A representative time-series of images are shown; cLVs were labeled red by the dextran injected into the cheek, while microspheres injected in the CM appear green; (inset) the white arrows show the direction of each microsphere show the direction of fluid motion. Scale bar: 50 μm b. The red outline represents the vessel wall detected using a custom MATLAB algorithm and tracked to obtain changes to vessel diameter synchronous with flow measurements. c. Rainbow-colored lines represent superimposed trajectories of tracked fluorescent microspheres flowing in the vessel. d. The time-averaged velocity field (green arrows) shows the net transport of lymph. e. Boxes indicate the local time-averaged fluid speed, which is slowest near vessel walls and fastest near the vessel center. f. Time-averaged velocity profiles measured at upstream, downstream, and at the valve indicate that highest centerline speeds are measured at the valve. Average flow speed profiles plotted as a function of distance from the left wall of the vessel. Colored lines in inset indicate the location of each profile. Scale bar: 50 μm. g. Reynold and h. Womersely numbers for the time averaged flow, mean±SEM, n=8 mice

FIGS. 2A, 2B, 2C, 2D, 2E, 2F, 2G, and 2H show intrinsic pump dysfunction in aging. a. Representative images of the local time-averaged flow speed shows that the lymph flow is nearly stagnant in aging. Scale bar: 50 μm b. Representative temporal variation of the median vessel diameter shows an age-dependent decrease in vessel function. Maxima and minima (diastolic and systolic peaks) are also indicated. c. Representative temporal variation of downstream velocity calculated from particle tracking for the different age groups shows the lymph flow speed decreased by aging. In (b) and (c), the dots represent the raw data, and the solid lines are obtained by smoothing over a 500-ms window. d. Intrinsic rate (frequency of contraction), e. Mean downstream speed and f. ejection rate (frequency of curves shown in b) exhibits an age-dependent decrease. g. Median vessel diameter has no change in aging. One-way ANOVA post hoc Tukey's test was performed (d-h), mean±SEM, n=5-7/group. h. Linear regression of intrinsic rate and ejection rate with 95% confidence intervals for all aging groups indicates a strong correlation between contraction of cervical lymph vessel and lymph efflux.

FIGS. 3A, 3B, 3C, 3D, 3E, and 3F show that lymphatic efflux volumes are reduced in old age. a. Temporal variation of the volume flow rate Q calculated from the product of the instantaneous spatially averaged downstream velocity and instantaneous cross-sectional lymphangion area. The lymphangion was assumed to have a circular cross-section, and the measured median vessel diameter was used to calculate the cross-sectional area. By definition, Q>0 signifies orthograde flow, and Q<0 signifies retrograde flow. b Retrograde fraction was increased by aging. C. Ejection Fraction, d. Ejection volume and e. Particle count was decreased by aging. One-way ANOVA post hoc Tukey's test was performed (b-e), mean±SEM, n=5-7/group. f. Representative images of valves (outlined by dotted lines) in young mice and old mice. Functional valves open and close to regulate flow in young mice but fail in old mice. Scale bar: 50 μm

FIGS. 4A, 4B, 4C, 4D, and 4E show that lymphatic smooth muscle actin loss impairs phasic contractions in old age. (a) Representative images of cervical lymph vessels after labeling for nuclei (DAPI), collagen IV (Col IV)²³ and smooth muscle actin^{24,25} Orthogonal sections show 3D distribution. Scale bar: 100 μm. (b-c) Collagen IV and SMA fluorescence vary transverse to the vessel direction. Vessel width was normalized to allow direct comparison between lymph vessels. Thick lines represent mean intensity with SEM shown as shadowed area, thin lines being individual animals. If more than one lymph vessel was collected per individual, average fluorescence was calculated. Color dots are individual animals. Mann-Whitney (Interanimal, Col IV: Old vs Young, p=0.3411, U=6; SMA: Old vs Young, p=0.0328, U=1). Unpaired Student's t test was performed, Bar graphs show area under the curve calculated for both proteins, as mean±SEM. Young (n=5), Old (n=4). (d) Representative phase-averaged expansion and contraction of lymph vessels for different age groups. Phase-averaging was performed over at least 10 cycles. (e) Representative normalized vessel wall velocity for different age groups, calculated by differentiating the curves in (d). By definition, positive speeds signify expansion, and negative speeds signify contraction.

FIGS. 5A, 5B, 5C, 5D, 5E, 5F, 5G, 5H, and 5I show that PGF_{2α} improves lymphatic function by promoting phasic vessel contractions. Representative images of a. time-averaged speed and b. velocity in young mice (top row) and 22-month-old mice (bottom row) before (left column) and after (right column) PGF_{2α} administration. c. Vessel diameter variation shows that PGF_{2α} stimulates the vessel and promotes intrinsic pulsation for young and aging groups, with a larger increase in intrinsic rate (e) and contraction amplitude ad (i) observed in old mice. d. Downstream velocity variation indicates that the PGF_{2α}-induced stimulation of vessel walls increased efflux, with faster mean downstream speeds (panel (f) and ejection rate (h). g. Median vessel diameter indicates that administration of PGF_{2α} reduces the size of the vessel. Paired t-test was performed in e-h, mean±SEM, n=5.

FIGS. 6A, 6B, 6C, 6D, 6E, and 6F show that PGF_{2α} restores lymphatic flow and reverses the effects of aging on CSF drainage. (a) Temporal variation of volume flow rate (Q) before and after administration of PGF_{2α} for young and old groups. b Retrograde fraction was decreased by PGF_{2α} for young and old group. c. Ejection Fraction, d. Ejection volume and e. Particle count was restored by PGF_{2α} in aging. Paired t-test was performed in b-e, mean±SEM, n=5. (f) Representative images of OVA-647 signals detected in cervical lymphatic vessels (CLV) before and after PGF_{2α} in young and old mice. (f) Progressive increase in the mean fluorescence intensity measured for 60 mins indicating increased efflux after PGF_{2α}. Quantification of mean pixel intensity (MPI) for the 60-minute in vivo imaging series depicted in f. Two-way ANOVA with Tukey's multiple comparisons test, n=6-7 mice/group. Scale bar: 2 mm.

FIGS. 7A, 7B, 7C, 7D, 7E, 7F, 7G, 7H and 71 show that cervical lymphatic efflux is not modulated by cardiac and respiration forces. (a-d) Representative vessel contractions (blue curves) and downstream velocity (orange) phase-averaged waveforms compared to changes in measured cardiac (red curves) and respiration signals (green curves). (e-f) Linear regression of intrinsic rate or ejection rate with 95% confidence intervals did not reveal any correlation with heart rate or respiration rate.

FIGS. 8A and 8B show that cardiac and respiration rates are unaffected by PGF_{2α}. a. Heart rate and b. Respiration rates before and after PGF_{2α} for young and old mice groups. Paired t-test was performed, mean±SEM, n=5.

FIGS. 9A, 9B, and 9C show that PGF_{2α} sustains lymphatic drainage into cervical lymph nodes. (a) Representative images of the ex vivo lymph nodes before and after PGF_{2α} administration in young and old mice group. Scale bar=2 mm. Fluorescent mean pixel intensity (MPI) normalized by the area of lymph nodes for SLN (b) and dLN (c). one-way ANOVA with post hoc Tukey's test, n=4/group. Bar graphs represent mean±SEM.

FIGS. 10A-10T show that aging causes loss of smooth muscle actin (a-SMA) coverage in scLVs. Representative images of collecting scLVs from (a) 2-month and (b) 18-month-old Prox1-GFP animals stained for α-smooth muscle actin (αSMA) and collagen 4a (Col4a). (c) Lymphatic endothelial cells (LEC) along the LV and within the lymphatic valve can be visualized with Prox1-GFP. (d) Smooth muscle cells (SMC) are αSMA-positive, and (e) Col4a is a marker of the collagen basement membrane. (f) Regions of interest (ROI) were divided into valvular and perivalvular ROIs. Quantification of the percentage of area covered for Prox1 at the valve (g) and the perivalvular region (h). Area covered for αSMA (i, j) and Col4a (k, l). (m) LECs were quantified as being DAPI+ and Prox1+. (n) Total number of DAPI+ and (o) DAPI+/Prox1+ cells do not decrease in 18-month-old animals compared to young. (p) SMCs were considered DAPI+/αSMA+ and were similarly abundant between young and old mice (q). (r) Orientation of αSMA and Col4a fibers. (s) Fiber orientation in degrees for αSMA and Col4a+ fibers in valvular and perivalvular ROIs. Orientation was computed as axial (0°) or longitudinal (+90° or −90°). (t) Coherence of fiber orientation as a percentage for fibers in valvular and perivalvular ROIs. Scale bars: 100 μm.

FIGS. 11A-11G show that PGF_{2α} reverses the effects of aging on CSF drainage and increases 780 brain clearance. (a) CSF clearance was evaluated via an intracisternal injection of ovalbumin-conjugated to Alexa 647 (OVA-Alexa 647), and CSF drainage was detected in cervical lymphatic vessels (cLV) with or without PGF2α in young and old mice. (b) Progressive increase in the mean fluorescence intensity measured for 60 mins, indicating increased efflux after PGF_{2α}. Scale bar: 2 mm. (c) Quantification of mean pixel intensity (MPI) for the 60-minute in vivo imaging series depicted in b. Two-way ANOVA with Tukey's multiple comparisons test, n=6-7 mice/group. Clearance of interstitial fluid (ISF) from the brain parenchymal was evaluated by injecting Direct Blue 53 (DB53) into the striatum. DB53 is cleared from the brain towards blood plasma, where it binds to circulating albumin and is retained for over 2 h, allowing stable quantification of brain clearance to plasma. (d) Coronal sections from anterior (A) to posterior (P), showing brain retention of DB53 after intrastriatal injection. Scale bar: 2 mm. (e) Quantification of brain retention expressed as % area of DB53 fluorescence. Unpaired Student's t test was performed. mean±SEM, n=6-7/group. (f) Representative imaging of the acquired in vivo femoral vein images after 1 μl 4% DB53 injection in the striatum. Scale bar: 1 mm. (g) Quantification of plasma clearance measured at the femoral vein. Two-way ANOVA with Sidak's multiple comparisons test. n=6-7 mice/group.

### DETAILED DESCRIPTION OF THE INVENTION

This disclosure relates to increasing glymphatic/lymphatic solute efflux from the brain or CNS. Certain aspects of this disclosure relates to restoration of cervical lymphatic vessel function. Such restoration in aging can rescue cerebrospinal fluid drainages.

### Cervical Lymphatic Vessels, Aging, and Cerebrospinal Fluid Drainage

Cervical lymphatic vessels (cLVs) have been shown to drain solutes and cerebrospinal fluid (CSF) from the brain. However, their hydrodynamical properties have never been evaluated in vivo. Certain aspects of this disclosure are based, at least in part, on an unexpected discovery that the major driver for cLVs flow is intrinsic pumping by contraction of the lymphatic vessel wall. As disclosed herein, 2-Photon Optical imaging with Particle Tracking In vivo of CSF tracers (2P-OPTIC) in cLVs of mice was developed to characterize their flow for the first time and it was identified that the major driver is intrinsic pumping by contraction of the lymphatic vessel wall.

Moreover, as disclosed herein, contraction frequency and flow velocity were reduced in aged mice which coincided with a reduction in smooth muscle actin. Slowed flow in aged mice could be rescued using topical application of an agonist of smooth muscle cells contractility, such as prostaglandin F_{2α} (PGF_{2α}), a prostanoid that increases smooth muscle contractility, which restored lymphatic function and increased net flow back to young levels. It is shown that cLVs are important regulators of CSF drainage and that restoring their function is an effective therapy for improving CSF clearance in aging.

Collecting LVs in the abdomen and the extremities drive flow through two mechanisms: active (intrinsic) and passive (extrinsic) pumping. Active pumping is generated by rapid, phasic contractions of the smooth muscle cells lining the lymphangion, the functional unit of LVs. Lymphangions are separated by one-way valves and their synchronized contractions expel lymph by limiting reflux and ensuring unidirectional flow. Passive pumping relies on the external compression of the LV by contraction of the surrounding skeletal muscle. The respiratory and cardiac cycle have also been proposed to propel lymph¹⁵. However, to date, nothing is known about the transport mechanisms of cLVs. Prior techniques include quantifying fluxes of labeled tracers that provides a transport flow rate over time but provide no insight into spatial properties of the flow. Ultrasound has been used to measure flow speed and vessel contractility but are unable resolve different tracers flowing within the lymph. Optical techniques have used relative changes in signal intensity of fluorescent dyes (indocyanine green or fluorescence-conjugated dextran) to measure lymphangion contractions but are unable to calculate flow speeds. An innovative approach has been optical imaging of particles flowing within the LVs of isolated vessels in vitro that have been artificially pressurized^{16 17}.

To overcome these limitations and obtain quantitative in vivo velocity data, inventors developed 2-Photon Optical imaging with Particle Tracking In vivo of CSF tracers (2P-OPTIC). This approached allowed high spatiotemporal resolution of flow speeds within a single lymphangion in vivo and used a dual tracer approach to enable simultaneous quantification of LV contractility and transport of CSF-derived solutes. As disclosed herein, contractions of cLVs are the primary motive force of fluid flow and these decline in aging coinciding with a partial loss of lymphatic smooth muscle cells. Furthermore, it was demonstrated that age-dependent decline of cLV function can be rescued by prostaglandin F_{2α} (PGF_{2α}), a smooth muscle contractile agent^{18,19}. Accordingly, age-induced impairment of cLV function could contribute to the development of neurodegenerative and neuroimmune diseases^{7 8}, recovery from acute injury, and blunted responses to CNS immunotherapy providing an opportunity for therapeutic intervention.

As disclosed herein, 2P-OPTIC, a new method to quantify LV flow dynamics, was introduced. These represent the first hydrodynamical measurements of cLVs of young and aged mice in vivo. It was found that cLVs are ~70 μm in diameter and contain flow speeds on the order of 150 μm/s. The primary driver of the viscous-dominated flows within these vessels are intrinsic contractions of the lymphangions and aging impairs cLV function by reducing intrinsic contractions. Similar to other lymphatic networks, inventors found a reduction in SMCs and their main contractile protein, smooth muscle actin. Additionally, it was found that dysfunctional valves in aging cLVs failed to properly regulate unidirectional transport of the slower flows, leading to large retrograde flows that exacerbated the inefficiencies in aging cLV. Moreover, as disclosed herein, the use of PGF_{2α}, a compound known to induce contractility of SMCs^{19 28} restores pumping of cLVs by increasing intrinsic contractions without changing cardiac and respiration rates. This disclosure shows that optimizing cLV function causes increased CSF drainage in young mice, and ultimately restored CSF clearance in old mice. Rescue of cLV function was validated both by a restoration in hydrodynamical transport of lymph and an increased rate of tracer accumulation in cervical lymph nodes.

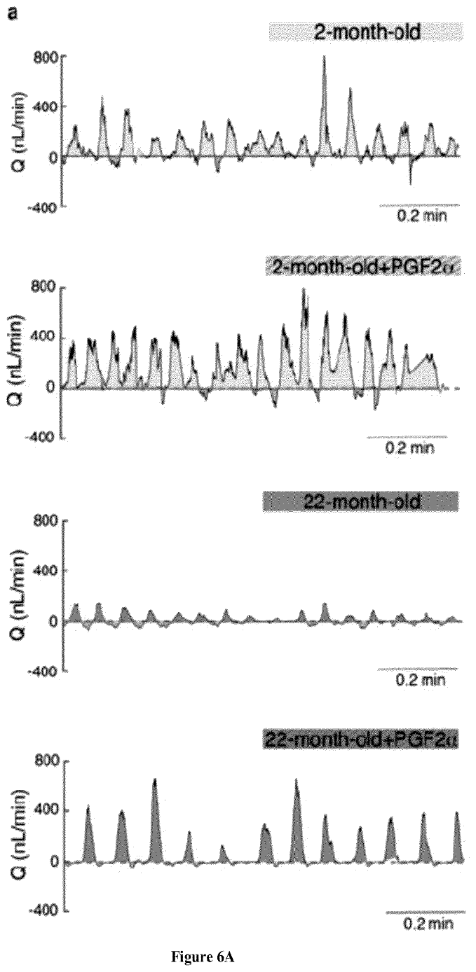

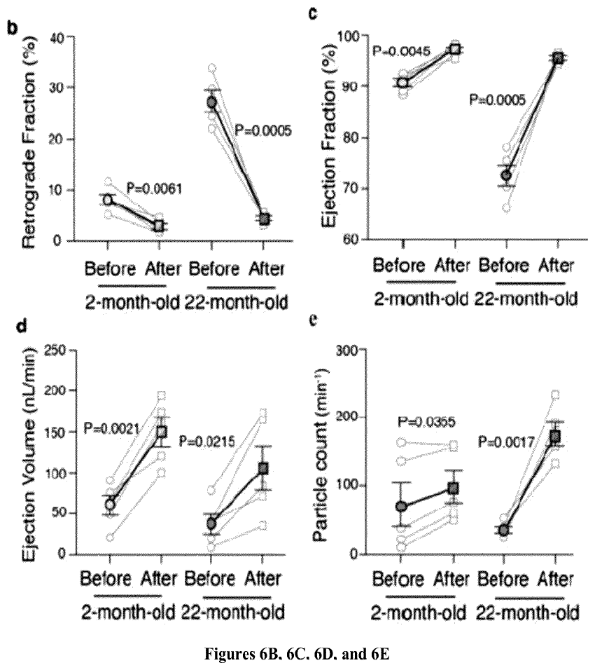

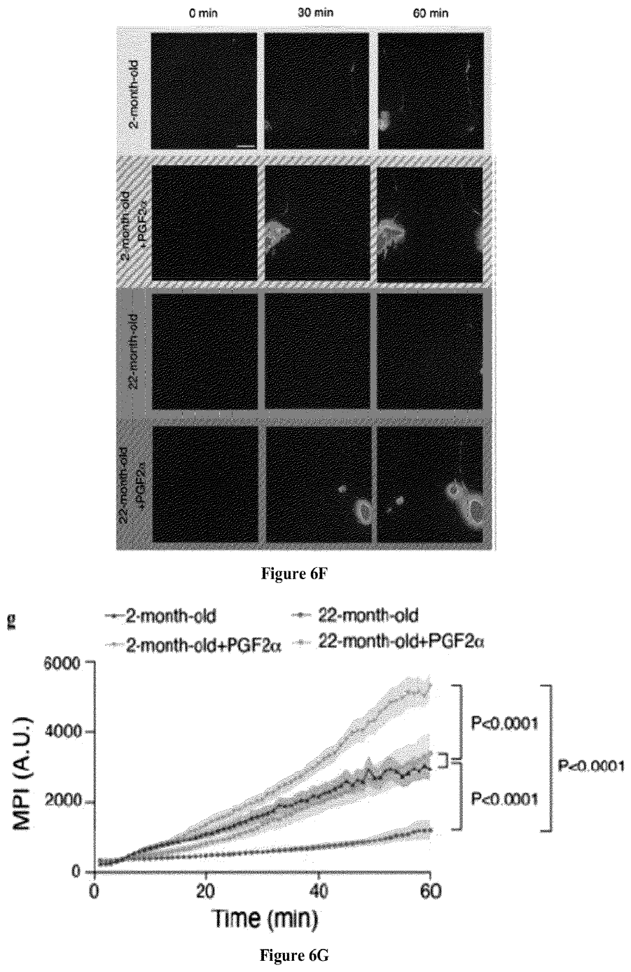

2P-OPTIC provides quantitative, real-time in vivo measurements of CSF drainage via cLVs. Prior studies have measured lymphatic flows in vivo relying on ultrasound-based measurements^{29,30}, optically tracking flowing lymphocytes^{22,31,32}, or used non-invasive optical coherence tomography (OCT)^{33,34} but none have evaluated cLV transport. Some studies have measured cLV kinetics using high-resolution stereomicroscopy to evaluate perineural outflow pathways and draining lymphatics of CSF^{5 35}, but while these methods allow large imaging volumes and robust tracer quantification (as seen in FIG. 6f) they do not provide quantitative information of lymph flow dynamics. Two-photon microscopy allows in vivo high spatiotemporal resolution imaging and optical sectioning enables accurate particle tracking velocimetry calculations. The dual tracer approach disclosed herein enabled the labeling of cLVs to track contraction dynamics and CSF to obtain lymph flow measurements with unprecedented detail. Disadvantages of this technique are the need to inject exogenous tracers and the surgical exposure of the superficial cervical LVs; however, this can be optimized by labelling endogenous components of lymph (i.e., cells or expressed proteins) and transdermal two-photon imaging. While 2P-OPTIC was developed for cLVs it can be employed in other lymphatic networks.

Unlike cardiovascular circulation, lymphatic networks lack a central pump to propel fluid. Therefore, lymphatic fluid is transported by intrinsic contractions of the lymphangions and by extrinsic contractions from surrounding skeletal muscle, breathing, and cardiac pulsations¹⁵. For flows in cLVs (both scLVs and dcLVs), inventors observed an average Re (<0.006) which is ~25% smaller than those measured in rat mesenteric LVs, but typical for small lymphatic vessels (<100 μm)³⁶. Flow speeds during contractions increased nearly two orders of magnitude (~1500 μm/s, instantaneous Re~0.6), which is also comparable to mesenteric lymphatic vessels³⁷. Even at the larger flow speeds, flow remains laminar and viscous dominated, while inertial effects have a small role. Wo in cLVs were ~0.03 indicating transient inertial effects are negligible and were an order of magnitude smaller than those in mesenteric vessels (0.1) and much smaller than those observed in the human thoracic duct (Wo=1.4), where inertial effects become important³⁸. Interestingly, Re and Wo measured in cLVs are comparable to those observed in similarly sized arterioles and venules, but also those seen in perivascular spaces of the glymphatic system³⁹. Cervical LVs transported about a third of the flow (~70 nL/min) of mesenteric vessels with a similar diameter (91 μm, ~230 nL/min)<. Flows were pulsatile and oscillated at a similar frequency (ejection rate) as the intrinsic contraction rate (~0.2 Hz≈12 min⁻¹) similarly to collecting LVs in the mesentery⁴⁰ but faster than abdominal LVs (flank, 6 min⁻¹).

The results disclosed herein show no effect of extrinsic sources (i.e., cardiac or respiratory cycles) since the dominant flow frequency was an order of magnitude smaller than that of either heart or respiration rates (2-3 Hz). That finding is intuitive, since the scLVs sit near the skin and are relatively unconstrained by the neck musculature that might otherwise cause extrinsic pumping. Extrinsic pumping might be more significant for flow in dcLVs since they are surrounded by several muscle groups in the neck. Nonetheless, as with venous drainage, cLVs can differ slightly from LVs in the extremities, given their position relative to the heart. This difference is likely more prominent in bipedal animals, but venous drainage in the head and neck of rodents also relies on gravity for venous return, compared to the periphery, where blood requires negative pressure from the cardiac cycle and extrinsic contractions from neighboring muscle groups to drive fluid against gravity back towards the heart. This feature and the data disclosed herein suggest that cLVs rely more on intrinsic contractions rather than extrinsic contractions to drive lymphatic flow.

Aging results in morphological (e.g., reduced lymphatic capillary density) and functional (e.g., reduced transport capacity of collecting LVs) alterations in LVs and all lymphatic networks studied to date including thoracic duct, skin, meningeal and mesenteric LVs have been shown to be impaired in aging⁴¹. The mechanisms underlying this decline are linked to lower production of lymphangiogenic factors and diminished regenerative capacity of lymphatic endothelial cells. Aging also leads to a reduction in the smooth muscle actin fiber coverage, both surrounding the membrane composition causing increased permeability and decreased contractility. The study here shows that cervical LVs also suffer from the same senescent smooth muscle actin-related changes seen in aging as other collecting lymphatic vessels in the rest of the body; yet this is an important observation because this collecting lymphatic chain affects CNS function.

As disclosed herein, cLVs are downstream facilitators of CSF drainage and represent an important component of the proposed glymphatic-lymphatic system. The glymphatic system transports intracranial CSF through perivascular spaces around arteries into the brain's extracellular space where it mixes with interstitial fluid and collects metabolic waste subsequently draining along perivenous spaces^{46 47}. Fluid is then collected by meningeal lymphatic vessels or exits along cranial and spinal nerves, where it is taken up by extracranial collecting LVs which all drain into the cLNs via cLVs^{48 49 50 5}. The organization of the glial-lymphatic pathway implies that cLVs are pivotal for CNS clearance⁴⁹, and this is highlighted by the fact that knocking out Prox-1 (thereby causing mispatterned and leaky LVs) and/or surgical ligation of cLVs leads to reduced CSF entry into the glymphatic pathway and ultimately slowed CSF clearance⁷. This indicates that a flow reduction downstream (lymphatics) leads to an upstream (glymphatic) slowing due to the conservation of mass, which is further supported by the fact that ligation can increase intracranial pressure⁹ and cause edema¹⁰. A similar effect was recapitulated by optically ablating meningeal lymphatics, a further upstream component of the pathway 7. While this disclosure demonstrates that aging drastically affects cLVs, there is evidence that aging also causes regression and plastic changes of meningeal LVs further contributing to delayed CSF drainage^{7 11}. In addition to lymphatic changes, aging is also associated with intracranial effects such as reduced CSF production at the choroid plexus62 and decreased CSF outflow from cranial foramina. Nonetheless, this disclosure shows that restoring cLV function alone is sufficient to rescue this age-induced effect, offering a new treatment strategy.

The therapeutic potential of reestablishing CSF clearance has been proposed for Alzheimer's disease, Parkinson's disease, ischemic stroke, and traumatic brain injury⁵¹. Meningeal LVs are also impaired in aged mice, but this effect can be rescued by treatment with vascular endothelial growth factor-C (VEGF-C), a lymphatic trophic factor, resulting in improved glymphatic flow and cognition in old mice⁷. This interaction is mediated by cLVs since their surgical ligation abolished the beneficial effect⁷. Ablating meningeal LVs increases amyloid-β burden and promotes deposition in mouse models of Alzheimer's disease⁷, while mice that lack meningeal lymphatics (K14-C-VEGFR-3-Ig transgenic mice) also retain higher amounts of tau⁵², whose presence correlates with Alzheimer's disease, like amyloid-β. Aged animals also exhibit a 40% decrease in the clearance intraparenchymally injected, radio-labelled amyloid-β. Similarly, ligation of cLVs restricted lymphatic efflux and exacerbated the aggregation of α-synuclein in mouse models of Parkinson's disease⁵³. This also extends to acute ischemic stroke, as mice with dysfunctional meningeal LVs have increased infarct volumes after transient middle cerebral artery occlusion⁵⁴. Besides an effect on disease progression, cLV function also modulates the effect of existing therapies. For example, meningeal LVs undergo extensive remodeling in mouse models of intracranial gliomas and metastatic melanoma resulting in decreased dendritic cell trafficking to cLNs dampening CD8+ responses and reducing the effect of anti-PD-1/CTLA-4 checkpoint therapy^{55 56}. Restoring LV function with VEGF-C improves the response to immune checkpoint inhibitors targeted against CNS tumors and anti-amyloid antibodies used in the treatment of Alzheimer's disease^{55 56 57}.

This disclosure shows that cLV can rescue both ISF and CSF drainage in aged mice by topical application of PGF_{2α} (dinoprost), a naturally occurring prostanoid, that binds to the prostaglandin F_{2α} receptor. An analogue of PGF_{2α} (carboprost) is already used clinically to induce uterine contractions and terminate postpartum bleeding. Carboprost is injected intramuscularly but could be adapted for transdermal administration to the neck region, which is relatively non-invasive compared to techniques targeting meningeal LVs that require VEGF-C gene therapy or hydrogel-encapsulated VEGF-C application to the skull (some of which are already in clinical trials).

Again, two-photon optical imaging with particle tracking in vivo of CSF tracers (2P-OPTIC) was developed and it was found that cervical lymphatic vessel pumping is impaired in aging. Aging led to a reduction in the frequency and amplitude of lymphatic contractions due to the loss of SMCs, resulting in slower flow speeds and lower ejected volumes. Furthermore, aged lymphatic valves failed to open and close properly, explaining the increase in retrograde flow. Since cLVs are points of convergence for multiple CSF outflow pathways the results disclosed herein provide evidence that improving cLV function can restore CSF drainage in aged animals and provide a therapeutic platform for treating a broad range of neurological diseases.

Accordingly, one aspect of this disclosure provides a method for improving glymphatic-lymphatic efflux from the CNS of a subject by among others enhancing the flow rate of a cLV of the subject.

In some embodiments, the enhancing the flow rate comprises increasing the contraction of the cLV. In some embodiments, the increasing the contraction of the cLV comprises administering to the subject an agonist of smooth muscle contraction. In some embodiments, the agonist comprises a cholinergic muscle stimulant. In some embodiments, the cholinergic muscle stimulant is selected from the group consisting of pyridostigmine, amifampridine, neostigmine, edrophonium, and ambenonium. In some embodiments, the agonist comprises a prostanoid. In some embodiments, the prostanoid comprises prostaglandin F_{2α} (PGF_{2α}).

In some embodiments, improving the glymphatic-lymphatic efflux or the flow rate of cLV from the CNS of a subject comprises administering to the subject an inhibitor of phosphodiesterase type 5a (PDE5 inhibitor).

In some embodiments, the agonist or inhibitor is administered to the neck of the subject. In some embodiments, the agonist or inhibitor is administered topically.

In some embodiments, the method further comprises one or more of the following: increasing influx of CSF through the CNS interstitium, and messaging the neck of the subject. In some embodiments, the increasing influx of CSF comprises administering to the subject an agent selected from the group consisting of a hypertonic solution a Stat-3 inhibitor, a bone morphogenetic protein (BMP) signaling axis molecule, an antagonist of AVP (vasopressin), an antagonist of atrial natriuretic peptide (ANP), an antagonist of Angiotensin II, an antagonist of AT2R receptors, and an antagonist of AT1 receptors. In some embodiments, the hypertonic solution comprises NaCl or mannitol.

Another aspect of the disclosure provides a method for promoting clearance of a waste product from the CNS interstitium, brain interstitium and/or spinal cord interstitium of a subject by among others improving glymphatic-lymphatic efflux from the CNS of the subject in the manner described above. In some embodiments, the waste product comprises amyloid β (Aβ), tau, or alpha synuclein. In some embodiments, the waste product comprises a drug or a metabolite thereof. Examples of the drug include small molecule compounds (e.g., chemotherapy compounds), biologics (e.g., proteins, antibodies, nucleic acids, and vectors), and others (e.g., liposome, nanoparticles).

A further aspect of this disclosure provides a method for treating a cerebral edema, a traumatic brain injury, a post-traumatic brain injury, or a neurodegenerative disease in a subject via among others improving glymphatic-lymphatic efflux from the CNS of the subject in the manner described above. In some embodiments, the neurodegenerative disease is Parkinson's disease (PD), Alzheimer's disease (AD), Alzheimer's disease with Lewy bodies, Lewy body dementia, mixed dementia, vascular dementia, frontotemporal dementia, chronic traumatic encephalopathy (CTE), HIV associated dementia, Lewy body disease, Huntington disease, or multisystem atrophy.

In some embodiments, the subject is a mammal. In some embodiments, the mammal is a human. In some embodiments, the mammal or human is an aged mammal or human.

### Agonists of Smooth Muscle Contraction

Certain aspect of this disclosure provides a method for improving glymphatic-lymphatic efflux from the CNS of a subject. The method comprises enhancing the flow rate of a cLV of the subject by increasing the contraction of the cLV. In some embodiments, the increasing the contraction of the cLV comprises administering to the subject an agonist of smooth muscle contraction.

As used herein an “agonist of smooth muscle contraction” refers to any agent that stimulates contraction of smooth muscle, such as that in the wall of the cLV. Examples of the agonist include a mamuscarinic agonist (e.g., carbachol), an al-adrenergic agonist (e.g., norepinephrine and propranolol), a K_{ATP} channel inhibitor (e.g., glibenclamide), a NOS inhibitor (e.g., NG-nitro-L-arginine (L-NNA)), a sGC blocker (e.g., ODQ), a PKA inhibitor (e.g., H-89 and KT5720), a COX-1 or 2 inhibitor (e.g., indomethacin), an anti-inflammatory drug such as NSAID (e.g., ketoprofen), a prostanoid (e.g., thromboxane A₂ (TxA₂) and PGF_{2α}, PGD₂, leukotrienes B4, C4 and D4, thromboxane A2 (TXA2) mimetic U46619), an EP4 receptor antagonist (e.g., AH23848B and GW627368x), an IP receptor antagonist (e.g., CAY10441), and cholinergic muscle stimulants (e.g., pyridostigmine, amifampridine, neostigmine, edrophonium, and ambenonium, and a derivative thereof, and/or a pharmaceutically acceptable salt thereof.

Prostanoids are a family of lipid mediators generated by the action of cyclooxygenase on a 20-carbon unsaturated fatty acid, arachidonic acid. Prostanoids comprise a family of lipid mediators formed from arachidonic acid (AA) via the prostaglandin (PG) H synthase or cyclooxygenase (COX) pathway. They include PGE₂, PGD₂, and PGF_{2α}, as well as prostacyclin (PGI₂) and thromboxane A₂ (TxA₂). Prostanoids, such as dinoprost and carboprost, have been known to modulate such biological processes as smooth muscle tone, vascular permeability, pain, fever, and platelet aggregation. Lymphatic contractility-promoting prostanoids (i.e., those promoting or inducing lymphatic contractility, pumping, or transport) are useful for the methods described herein.

Commercial therapies of pyridostigmine, amifampridine, neostigmine, edrophonium, and ambenonium are known in the art. Examples include the following:

| | |
|---|---|
| Pyridostigmine | MESTINON, MESTINON TIMESPAN, and REGONOL |
| Amifampridine | RUZURGI and FIRDAPSE |
| Neostigmine | PROSTIGMIN BROMIDE, PROSTIGMI, and BLOXIVERZ |
| Edrophonium | REVERSOL and ENLON |
| Ambenonium | MYTELASE |

As used herein, the terms “derivative,” “variant,” and “analogue” are used interchangeable to refer to a compound having a structure derived from the structure of a parent compound (e.g., a compound disclosed herein, e.g., prostanoid) and whose structure is sufficiently similar to those compounds disclosed herein and based upon that similarity, would be expected by one skilled in the art to exhibit the same or similar activities and utilities as the compounds, or to induce, as a precursor, the same or similar activities and utilities as the compounds. A derivative or analogue may be prodrug, ester, salt, or metabolite of the compound.

### Inhibitors of Phosphodiesterase Type 5a

Certain aspect of this disclosure provides a method for (i) improving glymphatic-lymphatic efflux from the CNS of a subject or/and (ii) enhancing the flow rate of a cLV of the subject by improving the relaxation of cLV smooth muscle via the inhibition of phosphodiesterase type 5a. For example, the method may comprise administering to the subject an inhibitor of phosphodiesterase type 5a or PDE5 inhibitor.

The terms “phosphodiesterase inhibitor” or “PDE inhibitor” refer to compounds and derivative or analogue (e.g., salts or solvates) thereof that function by inhibiting the activity of the enzyme phosphodiesterase. An exemplary phosphodiesterase is phosphodiesterase type 5 (PDE5). A PDE inhibitor can be a compound that decreases the activity of PDE in vivo and/or in vitro. PDE5 inhibitors increase cGMP levels by inhibiting the degradative action of PDE5 on cGMP.

Representative PDE 5 inhibitors include cyclic guanosine 3′,5′-monophosphate type five cGMP PDE inhibitors, also known as PDE5 inhibitors. PDE5 inhibitors include, for example, sildenafil, tadalafil, vardenafil, avanafil, lodenafil, udenafil, mirodenafil, zaprinast, P20066 (Ethypharm), SLx-2101 (Kadmon Pharmaceuticals), PF00489791 (Pfizer), INT007 (IntelGenx Technologies), and dasantafil. Additional exemplary PDE5 inhibitors include those described in U.S. Pat. Nos. 11,851,427; 9,387,210; 5,250,534; 5,859,006; 6,362,178; and 7,378,430; International Patent Publication Nos. WO/2008/095835, WO/2009/050554, WO/2009/124119, WO/2010/015589, WO/2010/074783, and WO/2011/015523; each of which is hereby incorporated by reference in its entirety.

Suitable effective dosages may include, but are not limited to about 0.0001 mg/kg to 100 mg/kg, 0.01 to 80 mg/kg, 0.01 to 50 mg/kg, 0.01 to 5 mg/kg, (e.g., 0.001 mg/kg, 0.002 mg/kg, 0.005 mg/kg, 0.01 mg/kg, 0.02 mg/kg, 0.05 mg/kg, 0.1 mg/kg, 0.2 mg/kg, 0.5 mg/kg, 1.0 mg/kg, 2.0 mg/kg, 5.0 mg/kg, 10 mg/kg, 15 mg/kg, 20 mg/kg, and 25 mg/kg), e.g., administered daily or on an as needed basis. A subject can be administered a therapeutic amount of a composition comprising a compound/agent at the following dose ranges: Sildenafil at a dose range of 25-100 mg/day), Tadalafil at a dose range of 2.5-20 mg/day or every other day), vardenafil at a dose range of 5-20 mg/day, or avanafil at a dose range of 50-200 mg/day.

### Compositions and Uses

Various compositions are available for the uses or therapies described herein, e.g. patches or injections; intramuscular injections, implants, oral tablets, subcutaneous formulations, intranasal formulations, buccal formulations, transdermal formulations such as the topical gels and solutions, or topical patches, and the like. In some embodiments, the composition can be a solid dosage formulation (e.g., tablet, capsule, granule, powder, sachet, or chewable), solution, gel, suspension, emulsion, shampoo, conditioner, cream, foam, gel, lotion, ointment, transdermal patch, film, tincture, or paste. Further provided herein are methods and uses of the compositions described herein for treating a disease, preventing a disease, treating a condition, and/or preventing a condition.

The formulation of the compound or derivative or analogue or salt thereof may provide a dose adequate to improve glymphatic-lymphatic efflux from the CNS. The pharmaceutically effective amount of the compounds, derivatives, analogues, or salts thereof present in the compositions as disclosed herein may depend on the patient's condition and the mode of administration.

Pharmaceutical compositions containing any of the compounds described herein or derivative or analogue or salt thereof may further comprise a pharmaceutically acceptable carrier. In certain embodiments, the pharmaceutical composition may be formulated (e.g., using the same excipients in the same ratios and/or comprising the same dose strength) or administrated in the same way as commercially available drugs, prodrugs, derivative products, including but not limited to MESTINON, MESTINON TIMESPAN, REGONOL, RUZURGI, FIRDAPSE, PROSTIGMIN BROMIDE, PROSTIGMI, BLOXIVERZ, REVERSOL and ENLON. The FDA-approved labels for each of these products are available at the website of the FDA, including with respect to their formulation, dosing, and administration.

The compounds and agents described above and related compositions are useful in methods of (i) improving glymphatic-lymphatic efflux from the CNS of a subject, (ii) promoting clearance of a waste product from the CNS interstitium, brain interstitium and/or spinal cord interstitium of a subject, and (iii) treating a cerebral edema, a traumatic brain injury, or a neurodegenerative disease in a subject.

In general, the compounds, agents, or compositions can be administered in a therapeutically effective amount by any of the accepted modes of administration. Suitable dosage ranges depend upon numerous factors such as the severity of the disease or condition to be treated, the age and relative health of the subject, the potency of the compound used, the route and form of administration, the indication towards which the administration is directed, and the preferences and experience of the medical practitioner involved. One of ordinary skill in the art of treating such diseases or conditions will be able, without undue experimentation and in reliance upon personal knowledge and the disclosure of this application, to ascertain a therapeutically effective amount of the compounds of the present disclosure for a given disease or condition. Thus, the compounds or compositions of the present disclosure can be administered as pharmaceutical formulations including those suitable for topical, oral (including buccal and sub-lingual), nasal, pulmonary, or parenteral (including intramuscular, intraarterial, intrathecal, subcutaneous and intravenous) administration or in a form suitable for administration by inhalation or insufflation.

A pharmaceutical composition described herein can be formulated to be compatible with its intended route of administration. Examples of routes of administration include parenteral (e.g., intravenous, intradermal, or subcutaneous), oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating an active compound or agent in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation are vacuum drying and freeze-drying which yields a powder of the active ingredient plus any additional desired ingredient from a previously sterile-filtered solution thereof.

Oral compositions generally can include an inert diluent or an edible carrier. For the purpose of oral therapeutic administration, the active compound or agent can be incorporated with excipients and used in the form of tablets, troches, or capsules, e.g., gelatin capsules. Oral compositions can also be prepared using a fluid carrier. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the active agent or compound can be delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration of a compound or agent can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds can be formulated into ointments, salves, gels, or creams as generally known in the art.

### Topical Use

In certain aspects, the manner of administration is topical or transdermal using a convenient daily dosage regimen which can be adjusted according to the degree of affliction. The term “topical,” as used herein, refers to the administration of a composition the skin and underlying tissues, as well as to administration to the mucosa and underlying tissues.

In particular, topical and/or transdermal treatment using the compounds, agents, or compositions disclosed herein is preferred for local control of a condition or disease state in a subject, while insuring that any unwanted side effects are minimized and curtailed. To that end, the pharmaceutical compositions of the present disclosure can be suitable for topical administration. In that case, the pharmaceutical compositions comprise one or more compounds or agents described herein, a pharmaceutically acceptable topical carrier, and optionally a permeation enhancer. In some aspects, the permeation enhancer can comprise a base. The base can be present at a concentration sufficient to provide a formulation pH in the range of approximately 7.5 to 13.0. The pharmaceutical composition can be aqueous. The aqueous pharmaceutical composition can be a cream, gel, lotion, paste, or solution.

Various skin-permeation enhancing agents are known in the art and can be used in this invention. Examples of suitable enhancers include, but are not limited to, ethers such as diethylene glycol monoethyl ether (available commercially as TRANSCUTOL) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80), and lecithin (U.S. Pat. No. 4,783,450); alcohols such as ethanol, propanol, octanol, benzyl alcohol, and the like; polyethylene glycol and esters thereof such as polyethylene glycol monolaurate (PEGML; see, e.g., U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids, particularly citric acid and succinic acid. AZONE® and sulfoxides such as DMSO and C₁₀ MSO may also be used.

Other suitable enhancers include those lipophilic co-enhancers typically referred to as “plasticizing” enhancers, i.e., enhancers that have a molecular weight in the range of about 150 to 1000, an aqueous solubility of less than about 1 wt. %, preferably less than about 0.5 wt. %, and most preferably less than about 0.2 wt. %. The Hildebrand solubility parameter of plasticizing enhancers is in the range of about 2.5 to about 10, preferably in the range of about 5 to about 10. Such enhancers are described in, e.g., U.S. Pat. No. 6,586,000, and WO 01/43775. Preferred lipophilic enhancers are fatty esters, fatty alcohols, and fatty ethers. Examples of specific and most preferred fatty acid esters include methyl laurate, ethyl oleate, propylene glycol monolaurate, propylene glycerol dilaurate, glycerol monolaurate, glycerol monooleate, isopropyl n-decanoate, and octyldodecyl myristate. Fatty alcohols include, for example, stearyl alcohol and oleyl alcohol, while fatty ethers include compounds wherein a diol or triol, preferably a C₂-C₄ alkane diol or triol, are substituted with one or two fatty ether substituents. Additional permeation enhancers are known in the art of topical drug delivery. See, e.g., Percutaneous Penetration Enhancers, Smith et al., editors (CRC Press, 1995).

A formulation described herein may be in any form suitable for topical application, for example to the skin, e.g., skin on the neck and surrounding tissues. It may comprise, for example, a cream, lotion, solution, gel, ointment, paste, plaster, paint, bioadhesive, or the like, and/or may be prepared to contain liposomes, micelles, and/or microspheres. Such a formulation may be aqueous, i.e., contain water, or may be nonaqueous and optionally used in combination with an occlusive overlayer so that moisture evaporating from the body surface is maintained within the formulation upon application to the body surface and thereafter.

Formulations of the invention may optionally contain a pharmaceutically acceptable viscosity enhancer and/or film former. A viscosity enhancer increases the viscosity of the formulation to inhibit its spread beyond the site of application. Balsam Fir (Oregon) is an example of a pharmaceutically acceptable viscosity enhancer. A film former, when it dries, forms a protective film over the site of application. The film inhibits removal of the active ingredient and keeps it in contact with the site being treated. An example of a film former that is suitable for use in this invention is Flexible Collodion, USP. As described in Remington, The Science and Practice of Pharmacy, 19th Ed. (Easton, Pa.: Mack Publishing Co., 1995), at page 1530, collodions are ethyl ether/ethanol solutions containing pyroxylin (a nitrocellulose) that evaporate to leave a film of pyroxylin. A film former may act additionally as a carrier. Solutions that dry to form a film are sometimes referred to as paints.

Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid. Preferred water-soluble ointment bases are prepared from polyethylene glycols of varying molecular weight; again, see Remington: The Science and Practice of Pharmacy for further information.

Creams, as also well known in the art, are viscous liquids or semisolid emulsions, either oil-in-water or water-in-oil. Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant.

As will be appreciated by those working in the field of pharmaceutical formulation, gels are semisolid, suspension-type systems. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the CARBOPOL. Also preferred are hydrophilic polymers such as polyethylene oxides, polyoxyethylene-polyoxypropylene copolymers, and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Lotions are preparations to be applied to the skin surface without friction, and are typically liquid or semiliquid preparations in which particles, including the active agent, are present in a water or alcohol base. Lotions are usually suspensions of solids, and preferably, for the present purpose, comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Pastes are semisolid dosage forms in which the active agent is suspended in a suitable base. Depending on the nature of the base, pastes are divided between fatty pastes or those made from a single-phase aqueous gels. The base in a fatty paste is generally petrolatum or hydrophilic petrolatum or the like. The pastes made from single-phase aqueous gels generally incorporate carboxymethylcellulose or the like as a base.

Plasters are comprised of a pasty mixture that is spread on the body, either directly or after being saturated into a base material such as cloth. Medications, including the bases of the invention, may be dissolved or dispersed within the plaster to make a medicated plaster.

Bioadhesives are preparations that adhere to surfaces of body tissues. Polymeric bioadhesive formulations are well known in the art; see, for example, Heller et al., “Biodegradable polymers as drug delivery systems,” in Chasin, M. and Langer, R., eds.: Dekker, New York, pp. 121-161 (1990); and U.S. Pat. No. 6,201,065. Suitable non-polymeric bioadhesives are also known in the art, including certain fatty acid esters (U.S. Pat. No. 6,228,383).

Formulations described in this invention may also be prepared with liposomes, micelles, and microspheres. Liposomes are microscopic vesicles having a lipid wall comprising a lipid bilayer, and can be used as drug delivery systems herein as well. Generally, liposome formulations are preferred for poorly soluble or insoluble pharmaceutical agents. Liposomal preparations for use in the instant invention include cationic (positively charged), anionic (negatively charged) and neutral preparations. Cationic liposomes are readily available. For example, N[1-2,3-dioleyloxy)propyl]-N,N,N-triethylammonium (DOTMA) liposomes are available under the tradename LIPOFECTIN®. (GIBCO BRL, Grand Island, N.Y.). Similarly, anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with DOTMA in appropriate ratios. Methods for making liposomes using these materials are well known in the art.

Micelles are known in the art to be comprised of surfactant molecules arranged so that their polar head groups form an outer spherical shell, while the hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethylammonium chloride, dodecylammonium chloride, polyoxyl 8 dodecyl ether, polyoxyl 12 dodecyl ether, nonoxynol 10 and nonoxynol 30. Micelle formulations can be used in conjunction with the present invention either by incorporation into a topical or transdermal delivery system, or into a formulation to be applied to a target site (e.g., vestibule) and surrounding tissues.

Microspheres, similarly, may be incorporated into the present formulations and drug delivery systems. Like liposomes and micelles, microspheres essentially encapsulate a drug or drug-containing formulation. Microspheres are generally, although not necessarily, formed from synthetic or naturally occurring biocompatible polymers, but may also be comprised of charged lipids such as phospholipids. Preparation of microspheres is well known in the art and described in the pertinent texts and literature.

Various additives known in the art may be included in the topical formulations. For example, solvents, including relatively small amounts of alcohol, may be used to solubilize certain formulation components. The present formulations may also include conventional additives such as opacifiers, antioxidants, fragrance, colorants, gelling agents, thickening agents, stabilizers, surfactants, and the like. Other agents may also be added, such as antimicrobial agents, to inhibit growth of microbes such as bacteria, yeasts, and molds. Exemplary antimicrobial agents are typically selected from the group consisting of the methyl and propyl esters of p-hydroxybenzoic acid (i.e., methyl and propyl paraben), sodium benzoate, sorbic acid, imidurea, and combinations thereof.

The compounds described above and related compositions are useful in methods of treating various disorders or conditions. Varieties or combinations of this therapy include, though are not limited to the following exemplary applications: a topical/transdermal spray using a radiating pump dispenser; a topical/transdermal salve/balm rubbed into the treated area; a topical/transdermal wound cleansing rinse; a topical/transdermal roll-on for pain relief, an impregnated mini-sponge individually hermetically sealed with said composition that can be reconstituted with water; a wound powder composed of micronized, freeze dried material, and a time-released epidermal/topical patch for staged and sequential delivery of said composition for site-specific application.

The therapeutic composition may preferably be administered as needed. For example, for severe conditions, about 1-4 times per day on a daily basis can be used. In addition, the therapeutic composition may alternatively be administered on a weekly, bi-weekly, tri-weekly, weekly or monthly basis until the condition is treated or remediated as desired. Furthermore, the administration may initially begin on a daily basis and then, in response to clinical improvement, transition to a weekly, monthly, etc. administration. Rather than being used solely as a treatment aid, the composition of the present invention may also be used to maintain a user in pain free condition.

In certain embodiments, the effective dose of a composition comprising one or more compounds/agents as described herein can be administered to a patient once. In certain embodiments, the effective dose of a composition can be administered to a patient repeatedly. Patients can be administered a therapeutic amount of a composition comprising a compound/agent at 0.0001 mg/kg to 100 mg/kg, such as 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg or 50 mg/kg. A composition comprising a compound/agent can be administered over a period of time, such as over a 5-minute, 10-minute, 15-minute, 20-minute, or 25-minute period. The administration is repeated, for example, on a regular basis, such as hourly for 3 hours, 6 hours, 12 hours or longer or such as biweekly (i.e., every two weeks) for one month, two months, three months, four months or longer. After an initial treatment regimen, the treatments can be administered on a less frequent basis. For example, after administration biweekly for three months, administration can be repeated once per month, for six months or a year or longer. Administration of a composition comprising a compound/agent can reduce levels of a marker or symptom of, for example, by at least 10%, at least 15%, at least 20%, at least 25%, at least 30%, at least 40%, at least 50%, at least 60%, at least 70%, at least 80% or at least 90% or more.

For certain administrations, the compositions can be provided in a suitable form containing about 0.001 to about 100 milligrams of active ingredient for the symptomatic adjustment of the dosage to the subject to be treated. An effective amount of the drug can be supplied at a dosage level of from about 0.0001 mg/kg to about 100 mg/kg of body weight per day. The compound or derivative or analogue or salt thereof may be provided in gel or cream forms in doses of 20 to 200 mg per day. In one embodiment, compound, derivative, analogue, or salt thereof are provided in a gel at doses of 50 to 100 mg/day, particularly 50 mg/day, 75 mg/day and 100 mg/day. Transdermal patches can used to deliver compound or derivative or analogue or salt thereof of 1 to 10 mg per day, particularly, 4 to 6 mg/day. The compound or derivative or analogue or salt thereof may also be provided by means of a buccal gel at a dose of 10 mg/day to 100 mg/day. In one embodiment, the dose can be a buccal gel is 40 to 80 mg/day. In one class of this embodiment, the dose can be 60 mg/day.

### Enhancing Influx

The methods described herein can further include enhancing glymphatic system influx through or to the CNS interstitium, brain interstitium and/or the spinal cord interstitium. One can enhance glymphatic system influx in a number of ways. For example, one can pump fluid through the central nervous system interstitium using methods and agents known in the art such as those described in WO2020072357 and WO2014130777, both of which are incorporated by reference. For instance, enhancing glymphatic system influx can comprise a step of administering an agent to a subject (such as a mammal) that increases glymphatic clearance, e.g., a Stat-3 inhibitor or BMP signaling axis molecules. In other embodiments, the agent is an antagonist of AVP (vasopressin) such as tolvaptan, conivaptan, or VPA-985, an antagonist of atrial natriuretic peptide (ANP) such as anantin, an antagonist of Angiotensin II such as losartan, an antagonist of AT2R receptors such as PD12331 9, or an antagonist of AT1 receptors such as valsartan. In another embodiment, the agent is an agent for use in the treatment of insomnia or as an aid for sleep, including but not limited to those listed below:

| Types of agent | Examples |
|---|---|
| Antihistamines | ALLEGRA ® (Fexofenadine), BENADRYL ® |
| | (Diphenhydramine), CLARITIN ® or TAVISTR (loratadine), |
| | CHLOR-T RIM ETON ® (chlorpheniramine maleate), |
| | DIMETANER (Brompheniramine, Phenylpropanolamine), and |
| | ZYRTEC ® (Cetirizine) |
| Nonprescription | Unisom Nighttime Sleep-Aid, Dormin, Nytol, Simply Sleep, |
| sleep aids | Sominex, Extra Strength Tylenol PM, Diphenhydramine |
| | hydrochloride, and Excedrin P.M. |
| Benzodiazepines: | PROSUM ® (estazolam), DALMANE ® (flurazepam), DORAL ® |
| | (quazepam), RESTORIL ® (temazepam), HALCION ® |
| | (triazolam), and VALIUM ® (diazepam) |
| Non- | Imidazopyridines: AMBIEN ®, AMBIEN ® CR. |
| benzodiazepines: | INTERMEZZO ® (Zolpidem) (class of its own), and SONATA ® |
| | (pyrazolopyrimidine) (class of its own) |
| | Melatonin receptor stimulator: ROZEREM ® (ramelteon), |
| | NOTED ® (chloral hydrate), PRECEDEX ® (dexmedetomidine |
| | hydrochloride), and LUNESTA ® (eszopiclone) |
| Barbiturates | NEMBUTAL ® (phenobarbital), |
| | and Amytal Sodium (amobarbital sodium), BUTISOL ® |
| | (butabarbital sodium), and SECONAL ® Sodium Pulvules |
| | (secobarbital sodium) |

In another embodiment, the agent can be an agent that prevents AQP4 depolarization or loss of AQP4 polarization, such as JNJ-1 7299425 or JNJ-17306861. In another embodiment, the step of increasing glymphatic influx comprises the step of pumping fluid through the central nervous system interstitium. Pumping can be accomplished by any device or method known in the art, for example, by using a mechanical pump, an infusion pump, etc.

Alternatively, the step of enhancing glymphatic system influx comprises administering a hypertonic agent to the subject. Preferably, the hypertonic agent is a hypertonic solution, which can be administered into plasma of the subject.

Each of the agents described above can be used alone or in combination with one or more of the other agents.

As used herein, “hypertonic” and “hypotonic” are relative terms e.g., in relation to physiological osmolality, but can diverge from this so long as the ultimate goal of an osmotic differential or gradient is achieved between two compartments (such as the blood plasma and the central nervous system interstitium) so as to promote the influx of glymphatic flow into central nervous system interstitium, brain interstitium and/or a spinal cord interstitium. Accordingly, a “hypertonic solution” refers any physiologically and/or pharmaceutically acceptable solution that is hypertonic with respect to physiological osmolality, including hypertonic saline or sugar solutions. As mentioned herein, hypertonic solutions preferred in this invention does not cause BBB disruption.

The methods described herein provide an agent (e.g., a pharmaceutical preparation) for injection that is hypertonic with respect to blood. To determine whether a pharmaceutical preparation is hypertonic with respect to blood, one calculates the osmolarity for all chemical components of a solution including the diluent. Tonicity can be calculated for fluids and dissolved or diluted medications, which are expressed in a numerical value of milliosmoles per liter of fluid (mOsm/L) or per kilogram of solvent (mOsm/kg). These two values also known as osmolarity and osmolality, respectively. The osmolarity of blood ranges between 285 and 310 mOsm/L and the osmolality of blood ranges between 275 and 299 mOsm/kg.

Solution osmolarity is based in part on the concepts of osmosis and osmotic pressure. Osmosis is the diffusion of solutes (dissolved particles) or the transfer of fluid through semipermeable membranes such as blood vessels or cell membranes. Osmotic pressure, which facilitates the transport of molecules across membranes, is expressed in osmolar concentrations and is referred to as hypo-osmotic (hypotonic), iso-osmotic (isotonic), or hyper-osmotic (hypertonic) when compared with biologic fluids such as blood or plasma. The term “tonicity” and “osmotic pressure” are often considered synonymous.

The osmotic pressure is the hydrostatic (or hydraulic) pressure required to oppose the movement of water through a semipermeable membrane in response to an ‘osmotic gradient’ (i.e., differing particle concentrations on the two sides of the membrane). Serum osmolality can be measured by use of an osmometer or it can be calculated as the sum of the concentrations of the solutes present in the solution.

As used herein, tonicity and osmotic pressure are to be considered synonymously, and are to be understood broadly. Tonicity can mean the effective osmolality and is equal to the sum of the concentrations of the solutes in a solution that have the capacity to exert an osmotic force across a membrane, including a cell membrane. In the strict sense, osmolality is a property of a particular solution and is independent of any membrane. Tonicity is a property of a solution in reference to a particular membrane. However, the disclosure shall refer to solutions being isotonic, hypertonic, or hypotonic with respect to biological solutions such as blood or plasma, and this referencing shall include the meaning that the particular solution is isotonic hypertonic, or hypotonic with blood or plasma with respect to a cell membrane of a cell in the blood or plasma or other biological solution.

An operational definition of tonicity can be used to explain the term. This can be based on an experiment of adding a test solution to whole blood and observing the result. If the RBCs in whole blood swell and rupture, the test solution is said to be hypotonic compared to normal plasma. If the RBCs shrink and become crenate, the test solution is said to be hypertonic compared to normal plasma. If the RBCs stay the same, the test solution is said to be isotonic with plasma. The RBC cell membrane can be the reference membrane. For example, whole blood placed in normal saline (i.e., 0.9% sodium chloride) will not swell, and hence normal saline is said to be isotonic.

The methods described herein include administering to a subject a pharmaceutical solution or preparation that is hypertonic with respect to plasma or blood. As hypertonic solutions, once injected into blood, may cause fluid shifts out of cells and a variety of negative effects, care should be taken to select a proper osmolality that are not so hypertonic as to cause significant thrombosis and/or vessel irritation. In one embodiment, the solution/preparation is considered to have suitable osmolality if 30 minute after injection into a subject in the manner described in the working example below, the resulting plasma osmolality is greater than about 320 mOsml·kg⁻¹ and less than about 600 mOsml·kg⁻¹, e.g., greater than about 340 or 350 and less than about 375, 400, 425, 450, 475, 500, or about 575 mOsml·kg⁻¹. In general, hypertonic solutions useful in this invention exhibit a tonicity that is greater than about 320 mOsml·kg⁻¹, e.g., 340 to 3,000 (e.g., 500 to 2,000, 1,000 to 2,000, 1,500 to 1,800) mOsml·kg⁻¹. Solutions with an osmolality that is greater than about 600 mOsml·kg⁻¹ should be used with care in injections.

Various primary bulking agents can be used for preparing a hypertonic solution/preparation for intravenous injection. Examples include ionizing agents, e.g., NaCl, and non-ionizing. Examples of non-ionizing bulking agents include, but are not limited to, mannitol, glycine, sucrose, lactose, other disaccharides, therapeutic proteins or the active ingredient of a formulation itself, or other bulking agents known to one skilled in the art. The concentrations of non-ionizing bulking agents do not significantly affect whether a solution has a sufficient ionic strength. However, their concentrations do have an effect on osmolarity, and therefore, their concentrations can have an effect on tonicity. In certain examples, NaCl or mannitol is used. The osmotic diuretic mannitol or hypertonic saline can establish an osmotic gradient between plasma and brain cells and draws water across the BBB into the vascular compartment. Exemplary dosages for mice were described in the working examples below. The human equivalent doses (HED) can be obtained using methods known in the art. See e.g., Nair A B, Jacob S. J Basic Clin Pharm. 2016 March; 7(2):27-31. doi: 10.4103/0976-0105.177703 and the FDA's Guidance for Industry. Estimating the Maximum Safe Starting Dose in Initial Clinical Trials for Therapeutics in Adult Healthy. For example, to a human subject, NaCl may be administered at 30 mg/kg or more (e.g., 30 to 300 mg/kg) and mannitol may be administered at 130 mg/kg or more (e.g., 130 to 1300 mg/kg).

### Therapeutic Methods

The methods and compositions described herein may be used for increasing glymphatic system efflux and interstitial waste clearance. Accordingly, the methods and compositions can be used for treating various related disorders.

In some embodiments, a method is provided for treating onset of a neurodegenerative disease in the brain and/or spinal cord (or CNS) of a subject comprising the step of increasing glymphatic-lymphatic efflux and/or clearance. In one embodiment of the method, reactive gliosis is reduced, thereby delaying or preventing onset of the neurodegenerative disease.

Reactive gliosis decreases or prevents interstitial waste clearance. Reactive gliosis decreases Aqp4-dependent bulk flow and reduces the volume of the extracellular space, impeding ISF solute clearance, including waste products, from the brain and spinal cord. Reactive gliosis is known in the art to be associated with neurodegenerative diseases such as Alzheimer's disease. Increasing gliosis is also observed in the aging mammalian brain. Reactive gliosis is also associated with certain autoimmune inflammatory disorders, notably multiple sclerosis. It has also been observed in the CNS of individuals suffering from amyotrophic lateral sclerosis (ALS). Thus, increasing glymphatic system efflux and/or clearance of waste products from the CNS can be used, in certain embodiments, to delay, prevent, decrease or reduce reactive gliosis and its neurodegenerative consequences. In another embodiment of the method, reactive gliosis is reduced, delayed or prevented. In another embodiment, the method comprises the step of administering a therapeutic agent to the subject that increases or promotes glymphatic system clearance.

A method is provided for promoting clearance of a waste product (e.g., a brain, spinal cord or CNS waste product) from the brain interstitium and/or spinal cord interstitium of a subject comprising the step of administering an agent to the subject that increases or promotes glymphatic efflux and/or clearance, whereby clearance of the waste product from the brain interstitium and/or spinal cord interstitium is promoted. The agent can be, for example, a diuretic. In some embodiments of the method, the brain, spinal cord or CNS waste product is amyloid β (Aβ) (e.g., soluble Aβ), tau or alpha synuclein. The method is also suitable for promoting clearance of virtually any brain waste product known in the art. In one embodiment, the method comprises the step of administering a therapeutic agent to the subject that increases or promotes glymphatic efflux and/or clearance.

The methods and compositions described herein cane be used for slowing, delaying or preventing accumulation of a brain waste product. Accordingly, a method is provided for slowing, delaying or preventing accumulation of a waste product in the central nervous system of a subject comprising the step of increasing glymphatic efflux, thereby increasing the clearance of the waste product from the central nervous system. In one embodiment, the method comprises the step of administering a therapeutic agent to the subject that increases or promotes glymphatic efflux. In an embodiment, the brain waste product is amyloid β (Aβ) (e.g., soluble Aβ) tau, or alpha synuclein. The method is also suitable for slowing, delaying or preventing accumulation of virtually any brain waste product known in the art. In a specific embodiment, a method is provided for decreasing, reducing, delaying onset of, or preventing amyloid β (Aβ), tau and/or alpha synuclein accumulation in brain interstitium of a subject. The method comprises the step of administering an agent to the subject that increases or promotes glymphatic efflux.

The methods and compositions described herein cane be used for increasing clearance of a therapeutic or modulatory agent from the brain interstitium of a subject. Accordingly, a method is provided for increasing clearance of a therapeutic or modulatory agent from the brain interstitium of a subject. The therapeutic or modulatory agent can be any known in the art, e.g., therapeutic or functionalized nanoparticle, chemotherapy agent, antineoplastic agent, immune modulator, antibody based therapeutic, viral vector, liposome or RNA-based therapeutic construct. In one embodiment, the method comprises the step of increasing glymphatic efflux.

In one embodiment, the method further comprises administering to the subject a drug for treating the neurodegenerative disease. Examples of drugs for treating Alzheimer's Disease may include cholinesterase inhibitors, N-methyl-D-aspartate (NMDA)-receptor antagonists, and monoclonal antibodies, such as, Donepezil, Galantamine, Rivastigmine, Memantine, the combination of Memantine and Donepezil, and Aducanumab. Examples of drugs for treating Parkinson's disease may include Suvorexant.

The patient or subject can be one having a neurological disorder or neurodegenerative disease, including, without limitation: Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, traumatic brain injury, etc.

In some embodiments, the neurological disorder is selected from: a neuropathy, an amyloidosis, cancer (e.g. involving the CNS or brain), an ocular disease or disorder, a viral or microbial infection, inflammation (e.g. of the CNS or brain), ischemia, neurodegenerative disease, seizure, behavioral disorder, lysosomal storage disease, etc.

Neuropathy disorders are diseases or abnormalities of the nervous system characterized by inappropriate or uncontrolled nerve signaling or lack thereof, and include, but are not limited to, chronic pain (including nociceptive pain), pain caused by an injury to body tissues, including cancer-related pain, neuropathic pain (pain caused by abnormalities in the nerves, spinal cord, or brain), and psychogenic pain (entirely or mostly related to a psychological disorder), headache, migraine, neuropathy, and symptoms and syndromes often accompanying such neuropathy disorders such as vertigo or nausea.

Amyloidoses are a group of diseases and disorders associated with extracellular proteinaceous deposits in the CNS, including, but not limited to, secondary amyloidosis, age-related amyloidosis, Alzheimer's Disease (AD), mild cognitive impairment (MCI), Lewy body dementia, Down's syndrome, hereditary cerebral hemorrhage with amyloidosis (Dutch type); the Guam Parkinson-Dementia complex, cerebral amyloid angiopathy, Huntington's disease, progressive supranuclear palsy, multiple sclerosis; Creutzfeld Jacob disease, Parkinson's disease, transmissible spongiform encephalopathy, HIV-related dementia, amyotropic lateral sclerosis (ALS), inclusion-body myositis (IBM), and ocular diseases relating to beta-amyloid deposition (i.e., macular degeneration, drusen-related optic neuropathy, and cataract).

Cancers of the CNS are characterized by aberrant proliferation of one or more CNS cell (i.e., a neural cell) and include, but are not limited to, glioma, glioblastoma multiforme, meningioma, astrocytoma, acoustic neuroma, chondroma, oligodendroglioma, medulloblastomas, ganglioglioma, Schwannoma, neurofibroma, neuroblastoma, and extradural, intramedullary or intradural tumors. For cancer, a neurological drug may be selected that is a chemotherapeutic agent.

Viral or microbial infections of the CNS include, but are not limited to, infections by viruses (i.e., influenza, HIV, poliovirus, rubella,), bacteria (i.e., Neisseria sp., Streptococcus sp., Pseudomonas sp., Proteus sp., E. coli, S. aureus, Pneumococcus sp., Meningococcus sp., Haemophilus sp., and Mycobacterium tuberculosis) and other microorganisms such as fungi (i.e., yeast, Cryptococcus neoformans), parasites (i.e., Toxoplasma gondii) or amoebas resulting in CNS pathophysiologies including, but not limited to, meningitis, encephalitis, myelitis, vasculitis and abscess, which can be acute or chronic.

Inflammation of the CNS includes, but is not limited to, inflammation that is caused by an injury to the CNS, which can be a physical injury (i.e., due to accident, surgery, brain trauma, spinal cord injury, concussion) and an injury due to or related to one or more other diseases or disorders of the CNS (i.e., abscess, cancer, viral or microbial infection).

Ischemia of the CNS, as used herein, refers to a group of disorders relating to aberrant blood flow or vascular behavior in the brain or the causes therefor, and includes, but is not limited to: focal brain ischemia, global brain ischemia, stroke (i.e., subarachnoid hemorrhage and intracerebral hemorrhage), and aneurysm.

Neurodegenerative diseases are a group of diseases and disorders associated with neural cell loss of function or death in the CNS, and include, but are not limited to: Parkinson's disease (PD), Alzheimer's disease (AD), Alzheimer's disease with Lewy bodies, Lewy body dementia, and mixed dementia, or associated with traumatic brain injury or ischemic (e.g., diffuse ischemic) brain injury, vascular dementia, frontotemporal dementia or chronic traumatic encephalopathy, adrenoleukodystrophy, Alexander's disease, Alper's disease, amyotrophic lateral sclerosis, ataxia telangiectasia, Batten disease, cockayne syndrome, corticobasal degeneration, degeneration caused by or associated with an amyloidosis, Friedreich's ataxia, frontotemporal lobar degeneration, Kennedy's disease, multiple system atrophy, multiple sclerosis, primary lateral sclerosis, progressive supranuclear palsy, spinal muscular atrophy, transverse myelitis, Refsum's disease, and spinocerebellar ataxia.

### Kit and Articles of Manufacture

In another aspect, this disclosure provides a kit or an article of manufacture containing materials useful for the methods described above. The article of manufacture comprises a container and a label or package insert on or associated with the container. Suitable containers include, for example, bottles, vials, syringes, solution bags, etc. The containers may be formed from a variety of materials such as glass or plastic. The container holds a composition which is by itself or combined with another composition effective (1) for improving delivery of a composition to a target site, (e.g., skin on the neck, central nervous system interstitium, brain interstitium and/or a spinal cord interstitium of a subject) or (2) for treating, preventing and/or diagnosing one or more of the conditions mentioned above. The container may have a sterile access port (for example, the container may be an intravenous solution bag or a vial having a stopper pierceable by a hypodermic injection needle). The label or package insert indicates that the composition is used for treating the condition of choice.

Moreover, the article of manufacture may comprise (a) a first container with a composition contained therein, wherein the composition comprises an agent that enhances glymphatic system efflux and (b) a second container with a composition contained therein, wherein the composition comprises an agent that enhances glymphatic system influx. The article of manufacture may comprise a third container with a composition contained therein, wherein the composition comprises a therapeutic agent or imaging agent.

The article of manufacture in this embodiment of the invention may further comprise a package insert indicating that the compositions can be used to treat a particular condition. Alternatively, or additionally, the article of manufacture may further comprise a fourth container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In some embodiments, the kit or article of manufacture further comprises instructional materials containing directions (i.e., protocols) for the practice of the methods described herein (e.g., instructions for using the kit for administering a composition). While the instructional materials typically comprise written or printed materials, they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD-ROM), and the like. Such media may include addresses to internet sites that provide such instructional materials.

### Definitions

“Prodrug” or “pharmaceutically acceptable prodrug” refers to a compound that is metabolized, for example hydrolyzed or oxidized, in the host after administration to form any of the compounds of the present disclosure. The present disclosure includes within its scope, prodrugs of the compounds described herein. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C1-C8 alkyl, C2-C8 alkenyl, C2-C8 alkynyl, aryl, C7-C12 substituted aryl, and C7-C12 arylalkyl esters of the compounds described herein may be preferred. Conventional procedures for the selection and preparation of suitable prodrugs are described, for example, in “Design of Prodrugs” Ed. H. Bundgaard, Elsevier, 1985. As used herein, “prodrug” may also refer to a naturally occurring precursor of a drug.

The term “biologically active metabolite” means a pharmacologically active product produced through metabolism in the body of a specified compound as disclosed herein or salt thereof.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “effective amount” as used herein refers to the amount of an agent needed to alleviate at least one or more symptom of the disease or disorder, and relates to a sufficient amount of pharmacological composition to provide the desired effect. The term “therapeutically effective amount” therefore refers to an amount of the agent that is sufficient to provide a beneficial effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

The terms “decrease,” “reduced”, “reduction”, and “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. A decrease can be preferably down to a level accepted as within the range of normal for an individual without a given disorder.

The terms “improve,” “increased”, “increase”, “enhance”, or “activate” are all used herein to mean an increase by a statically significant amount. In some embodiments, the terms “improve,” “increased,” “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” or “individual” means a human or animal. Usually the animal is a vertebrate such as a primate, rodent, domestic animal or game animal. Primates include chimpanzees, cynomolgous monkeys, spider monkeys, and macaques, e.g., Rhesus. Rodents include mice, rats, woodchucks, ferrets, rabbits and hamsters. Domestic and game animals include cows, horses, pigs, sheep, goats, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a human or a non-human mammal. The mammal can be a human, non-human primate, mouse, rat, dog, cat, horse, or cow, but is not limited to these examples. Mammals other than humans can be advantageously used as subjects that represent animal models of disorders. The terms, “individual,” “patient” and “subject” are used interchangeably herein. A subject can be male or female.

A subject can be one who has been previously diagnosed with or identified as suffering from or having a condition or disorder in need of treatment or one or more complications related to such a condition or disorder, and optionally, have already undergone treatment for such a condition or disorder or the one or more complications related to the condition or disorder. Alternatively, a subject can also be one who has not been previously diagnosed as having a condition or disorder or one or more complications related to the condition or disorder. For example, a subject can be one who exhibits one or more risk factors for the condition or disorder or one or more complications related to the condition or disorder or a subject who does not exhibit risk factors.

A “subject in need” of treatment for a particular condition or disorder can be a subject having that condition or disorder, diagnosed as having that condition or disorder, or at risk of developing that condition or disorder.

As used herein, the term “administering,” refers to the placement of an agent as disclosed herein into a subject by a method or route which results in at least partial delivery of the agent at a desired site. Pharmaceutical compositions comprising the agents disclosed herein can be administered by any appropriate route which results in an effective treatment in the subject. The terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, ophthalmic administration, intraaural administration, intracerebral administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder associated with a disorder. Treatment is generally “effective” if one or more symptoms or clinical markers are reduced. Alternatively, treatment is “effective” if the progression of a disease is reduced. That is, “treatment” includes not just the improvement of symptoms or markers, but also a slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

A “therapeutically effective amount” is an amount sufficient to remedy a disease state or symptoms, particularly a state or symptoms associated with the disease state, or otherwise prevent, hinder, retard or reverse the progression of the disease state or any other undesirable symptom associated with the disease in any way whatsoever.

A “prophylactically effective amount” is an amount of a pharmaceutical composition that, when administered to a subject, will have the intended prophylactic effect, e.g., preventing or delaying the onset (or reoccurrence) of the disease state, or reducing the likelihood of the onset (or reoccurrence) of the disease state or associated symptoms. The full therapeutic or prophylactic effect does not necessarily occur by administration of one dose, and may occur only after administration of a series of doses. Thus, a therapeutically or prophylactically effective amount may be administered in one or more administrations.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier or excipient” refers to a carrier medium or an excipient which does not interfere with the effectiveness of the biological activity of the active ingredient(s) of the composition and which is not excessively toxic to the host at the concentrations at which it is administered. In the context of the present invention, a pharmaceutically acceptable carrier or excipient is preferably suitable for topical formulation. The term includes, but is not limited to, a solvent, a stabilizer, a solubilizer, a tonicity enhancing agent, a structure-forming agent, a suspending agent, a dispersing agent, a chelating agent, an emulsifying agent, an anti-foaming agent, an ointment base, an emollient, a skin protecting agent, a gel-forming agent, a thickening agent, a pH adjusting agent, a preservative, a penetration enhancer, a complexing agent, a lubricant, a demulcent, a viscosity enhancer, a bioadhesive polymer, or a combination thereof. The use of such agents for the formulation of pharmaceutically active substances is well known in the art (see, for example, “Remington's Pharmaceutical Sciences”, E. W. Martin, 18th Ed., 1990, Mack Publishing Co.: Easton, PA, which is incorporated herein by reference in its entirety).

A “neurological disorder” refers to a disease or disorder which affects the CNS and/or which has an etiology in the CNS. Exemplary CNS diseases or disorders include, but are not limited to, neuropathy, amyloidosis, cancer, an ocular disease or disorder, viral or microbial infection, inflammation, ischemia, neurodegenerative disease, seizure, behavioral disorders, and a lysosomal storage disease. Specific examples of neurological disorders include, but are not limited to, neurodegenerative diseases (including, but not limited to, Lewy body disease, postpoliomyelitis syndrome, Shy-Draeger syndrome, olivopontocerebellar atrophy, Parkinson's disease, multiple system atrophy, striatonigral degeneration, tauopathies (including, but not limited to, Alzheimer disease and supranuclear palsy), prion diseases (including, but not limited to, bovine spongiform encephalopathy, scrapie, Creutzfeldt-Jakob syndrome, kuru, Gerstmann-Straussler-Scheinker disease, chronic wasting disease, and fatal familial insomnia), bulbar palsy, motor neuron disease, and nervous system heterodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), cancer (e.g. of the CNS, including brain metastases resulting from cancer elsewhere in the body).

As used herein, the term “a,” “an,” “the” and similar terms used in the context of the present invention (especially in the context of the claims) are to be construed to cover both the singular and plural unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to within 10%, preferably within 5%, and more preferably within 1% of a given value or range. Alternatively, the term “about” refers to within an acceptable standard error of the mean, when considered by one of ordinary skill in the art.

As used herein the term “comprising” or “comprises” is used in reference to compositions, methods, and respective component(s) thereof, that are essential to the method or composition, yet open to the inclusion of unspecified elements, whether essential or not. The term “consisting of” refers to compositions, methods, and respective components thereof as described herein, which are exclusive of any element not recited in that description of the embodiment. As used herein the term “consisting essentially of” refers to those elements required for a given embodiment. The term permits the presence of elements that do not materially affect the basic and novel or functional characteristic(s) of that embodiment.

### EXAMPLES

### Example 1

This example describes material and methods used in Examples 2-7 bellow.

### Animals

The University Committee on Animal Resources of the University of Rochester Medical Center (Protocol No. 2011-023) approved all the experiments. Male C57BL/6 mice, 8-week-old of age were acquired from Charles River Laboratories (Wilmington, MA, USA). 18-month and 22-month-old were acquired from the National Institute on Aging (Bethesda, MD, USA).

### Drugs

Anaesthesia was administered with ketamine/xylazine (100/10 mg kg-1, intraperitoneally). PGF_{2α} (5 μM in PBS, VWR) was directly local administrated onto the cervical lymph vessel.

### Surgical Procedure

Anesthetized mice were placed in a stereotaxic frame and a 30G needle connected to PE-10 tubing filled with artificial (aCSF) was inserted into the cisterna magna as described here. Then, animals were positioned on their backs on a heating pad to maintain body temperature. A midline surgical incision was made from the chin to the sternum to expose the superficial cervical lymph vessel. To expose deep cervical LVs, the superficial cervical fascia was incised and deep neck dissection along the lateral border of the trachea was achieved by separating along the medial border of sternocleidomastoid and posterior belly of the digastric muscles to exposes the common carotid artery and internal jugular vein. Deep cervical LVs run superficial to both these vascular structures and were confirmed as being Prox1-GFP positive. Afterwards, FITC-Dextran (3 kDa, 1% solids in saline, 20 μl, Invitrogen) was injected in each cheek. Red fluorescent polystyrene microspheres (FluoSpheres™ 1.0 μm, 580/605 nm, 0.25% solids in artificial CSF (aCSF), Invitrogen) were briefly sonicated and infused at 2 μL min-1 for 5 min into the cisterna magna catheter with a syringe pump (Harvard Apparatus).

### Measurement of Vital Signs

Heart and respiratory rate were measured using a small animal physiological monitoring 494 device (Harvard Apparatus) which acquired measurements at 1 kHz and 250 Hz, respectively. The signals were digitized and recorded with a DigiData 1550A digitizer and AxoScope software 496 (Axon Instruments).

### In Vivo Two-Photon Laser Scanning Microscopy

A resonant scanner B scope (Thorlabs) with a Chameleon 498 Ultra II laser (Coherent) was used for two-photon imaging. FITC-dextran and red microspheres were excited at a 820 nm wavelength. A water immersion 20× objective (1.0 NA, Olympus) was used. Images were acquired at 60 Hz (ThorImageLS software) simultaneously with physiological recordings (3 kHz, ThorSync software).

### Image Processing

16-bit images were obtained from two-photon microscopy using two channels (red and green), each with spatial dimensions of 256×256 at least. The green channel captured the FITC-dextran in the lymph vessel, while the red channel captured the fluorescent microspheres flowing in the lymph vessel. Image registration was the first step in imaging processing, necessary to account for movement of the mouse in the background. An efficient algorithm in MATLAB was used to apply rigid translations (no rotation or deformation) that were calculated to an accuracy of 0.2 pixel. At least 10 continuous images from the recording that showed the most stability and least movement were used as reference for the rigid translations. Erroneous correlations in the translations (where the algorithm failed) were inspected and manually corrected via linear interpolation. The corrected translations were then sequentially applied to the images whose edges are also padded with zero-value pixels to ensure spatial homogeneity across all images without modifying the image resolution. Particles were then detected in each image by applying a minimum intensity threshold. These particles are typically resolved across 3-4 pixels in the image with an image resolution of 1.29 μm/pixel. Motion artifact index was calculated from time-lapse imaging using an epifluorescent macroscope. Dissimilarity between sequential images was computed as the mean of the absolute value of the difference of each pixel.

### Particle Tracking Velocimetry

The particles detected in each image were tracked using an automated PTV routine implemented in MATLAB 78,79. Briefly, the algorithm locates each particle with a sub-pixel accuracy and correlates its movement across images to obtain a series of particle locations (particle tracks) for the duration of the recording. The particle velocities are then calculated by convolution with a Gaussian smoothing and differentiation kernel. Stagnant particles that had adhered to the wall of the lymph vessel, and hence no longer follow lymph flow, were masked in each image by subtracting a dynamic background image. This image was different for each frame and was computed by taking the average of 100 frames before and after the given image.

### Lymph Vessel Diameter and Contraction Frequency

Vessel diameter: Using a custom MATLAB code, the median vessel diameter was measured for the same temporal segments used for speed measurement. First, the centreline of the imaged vessel was identified by an algorithm that applied a spatially varying threshold for each image, then identified the edge, using the “skeletonize” function in MATLAB. The identified centreline was also verified manually. 20 transverse lines were then interpolated and the changes in the vessel diameter were measured with sub-pixel accuracy by identifying locations along the transverse lines where the pixel intensity dropped to 20-40% of the maximum value. Then, the median over space and time was computed. Any small systematic drift in the median value (due to translation of the two-photon imaging plane or movement in and out of the imaging plane due to respiration artifacts) was smoothed by computing a moving average over a 1 second window.

Intrinsic pulsation rate: The MATLAB function “findpeaks” was used to identify the maxima and minima of the smoothed vessel diameter measurements obtained above. Contractions of amplitude less than 3% of the mean diameter or those separated by less than 2 s were rejected. The mean values of maxima and minima were then identified in 4-5 one-minute time windows and the average value of the two means was assigned as the contraction frequency per minute.

### Computing Flow-Derived Quantities

Mean clearance speed: Five-minute recording segments were manually selected for analyses to ensure uninterrupted continuous particle count in each frame. Mean clearance speeds were obtained by segregating the imaged domain into bins of size 5 or 10 pixels (depending on particle counts), computing the time-averaged flow velocity in each bin, and then computing the spatial average. Inventors used at least 30 separate measurements in space with each at least 7 measurements in each bin to ensure reliable estimates of the mean.

Downstream velocity: the spatially averaged mean downstream velocity, V_{downstream}, was computed in each frame using the relationship V_{downstream}=u·ū_{avg}, where u is the instantaneous particle velocity and ū_{avg} is the field of unit vectors computed from the time-averaged flow field in the direction of lymphatic fluid efflux from vessel.

Volumetric flow, ejection fraction, retrograde fraction, and net efflux volume: The volumetric flow rate, Q, for each frame was computed as the product of the measured downstream velocity, V_{downstream} and approximate cross-sectional area of the vessel, (πD²/4, where D is the measured median vessel diameter in each frame. A positive value of Q indicates lymphatic fluid ejection from vessel (orthograde flow) and a negative value indicates retrograde flow. The time profile of volumetric flow rate was computed for one-minute time windows in the recording. The area under this curve was computed in MATLAB to derive volume flow rate related quantities. The total positive area indicates the volume of lymph ejected from the vessel (shaded in green) and the negative area (area of curve below zero) indicates the volume of lymph that flows into the lymphangion upstream of the imaged section. The ejection fraction is calculated as the percentage of the flow in the forward direction (the ratio of total positive flow volume shaded in green to the total volume flowing through lymphangion) and the retrograde fraction is calculated as the percentage of flow volume that flows upstream (the ratio of total volume shaded in red to the total volume flowing through lymphangion). The net volume cleared from the vessel was difference between the total positive and negative flow volumes in a one-minute window.

### Immunohistochemistry

Mice were transcardially perfused with 4% paraformaldehyde in 0.1 M phosphate buffer, pH 7.4, and the cervical lymph vessels were carefully dissected under a dissection macroscope and placed over gelatin-KCr(SO4)2 coated slides. The vessels while still attached to their respective nodes were incubated in 24-well microtiter plates (Corning) in blocking solution of 10% serum-PBS containing 0.1% Triton X-100, 0.05 mg/mL Affinipure Fab fragment (Jackson ImmunoReseach, West Grove, PA, US), 0.2% gelatin for 2 h at room temperature, followed by three 5-minute washes in PBS. The vessels were incubated using the mesh well inserts that allowed the entire sample (vessels attached to the nodes) to be incubated and washed by blocking solution.

Incubations with the primary antibodies for smooth muscle actin (1:250, A5228, Millipore Sigma), collagen IV (1:400, 2150-1470, BioRad), lymphatic endothelial cell marker—LYVE1—(1:400, 25-0443-82, Invitrogen) were carried out overnight at 4° C. in PBS containing 5% normal donkey serum, 0.1% Triton X-100 and 0.2% gelatin under agitation. After PBS washing, tissues were incubated with a mixture of secondary antibodies Cy3-conjugated donkey anti-rabbit, Cy-2 conjugated donkey anti-rabbit, and Cy5-conjugated donkey anti-mouse (1:500; Jackson ImmunoResearch; catalog 711165152 and 715175151, respectively) and DAPI (2.5 μg/mL, D21490, Invitrogen) in PBS containing 5% normal donkey serum, 0.1% Triton X-100 and 0.2% gelatin for 1 hour at room temperature. PBS-washed samples were then mounted on microscope slides in wells created with adhesive spacers. The lymphatic vessels were unfurled from the respective nodes and were mounted with ProLong™ Gold Antifade Mount (P36930, Invitrogen).

The immunolabeled lymph vessels were imaged using confocal microscopy (FV3000RS, Olympus, MA, US). Multichannel Z-stacks (20×, 1 μm step size) across the total width of the lymph nodes were acquired using the same acquisition parameters for all samples. To evaluate protein distribution, the central plane of the acquired Z-stacks was analysed using the plot profile function of Image J (version 1.53c, NIH, Bethesda, MD, USA), drawing a 200 μm wide line perpendicular to the vessel lumen, obtaining the linear distribution of fluorescence. Due to the variability of the lymph vessel width, the X axis of the obtained graphs was normalized by its own diameter. The resulting distributions had the raw fluorescence distribution on the Y axis and width ratio on X axis, allowing their direct comparison. To evaluate differences in protein expression, the area under the curve was calculated for all samples and both proteins. Coverage area was calculated by using an automated thresholding step and computing the percent of pixels within a lymphatic vessel region of interest (ROI) that was manually drawn. Prox1-GFP images were used to generate an ROI around the valve and the prevalvular and postvalvular regions of the LV were combined to generate a perivalvular ROI. DAPI+ nuclei were manually counted using CellCounter on Fiji. To quantify lymphatic endothelial cells (LEC), DAPI+ cells that were also Prox1+ were considered LECs. LECs were expressed as a percent of all DAPI+ nuclei. Smooth muscle cells (SMC) were quantified from DAPI+ channel images where LEC nuclei were subtracted. Remaining DAPI+ positive nuclei that also stained positive for SMA were considered SMCs and expressed as % of DAPI+ nuclei. Fiber orientation was computed using OrientationJ on Fiji. Since orientations ranged from −90 to 900 but functionally have a longitudinal orientation, we calculated the absolute value of all negative orientations and expressed orientation in a range from 0-90°. The OrientationJ plugin also calculates coherence of fibre orientation, and this was also evaluated for both valvular and perivalvular ROIs. Representative videos were generated from confocal high magnification Z-stacks (60×, 0.4 μm step size, FV3000RS, Olympus, MA, US) acquired with identical parameters.

### Epi-Fluorescent Optical Microscopy

Ovalbumin-conjugated to Alexa Fluor 647 (OVA-647, 0.5% w/v, 45 kDa, Invitrogen, 10 μL, 2 μL/min) was injected into the cisterna magna through a 30G needle connected to PE-10 tubing using a syringe pump (Harvard Apparatus). Cervical lymphatic vessels were surgically exposed as before and imaged using a fluorescent macroscope (MVX10, Olympus) with a PRIOR Lumen 1600-LED light source and ORCA Flash 4.0 digital camera (Hamamatsu) using Metamorph software. The images at 20× magnification from the far-red emission channel (647 nm) were collected at 1 min intervals for 60 min following the start of the injection. Exposure time was kept constant across all the experimental groups. One microliter PBS with or without 5 μM PGF2a was applied directly onto the cervical lymph vessel simultaneously with the injection of OVA-647 into cisterna magna.

### Lymph Node Imaging

One microliter PBS with or without 5 μM PGF2a was applied directly onto the cervical lymph vessel simultaneously with the injection of OVA-647 (0.5% w/v, 45 kDa, Invitrogen, 10 μL, 2 μL/min) into cisterna magna for 90 min. Then the superficial and deep cervical lymph nodes were dissected and put on the microscope slices (Fisherbrand). The lymph nodes were placed under the macroscope (MVX10 Research Macro Zoom Microscope, 633 Olympus). The images were acquired in the far-red emission channel (647 nm).

### Clearance Assay

Clearance assay was conducted using our published method (Pla, V. et al. A real-time in vivo clearance assay for quantification of glymphatic efflux. Cell Rep 40, 111320 (2022)). Briefly, anesthetized mice were mounted in a stereotactic apparatus and a small burr hole was made (AP+0.6 mm; ML—2.0 mm). A guide cannula (26G, C315G SPC, 4.5 mm below pedestal) and dummy cannula (33G, C315DC/SP, 0.1 mm projection) was placed at DV 3.3 mm. After 48 hr, the dummy cannula was replaced by the inner cannula (33G, C3151/SP, 0.1 mm projection), connected by a PE10 tubing to a 10 μL Hamilton syringe containing 4% (w/v) DB53 in sterile aCSF. The left femoral vein was exposed via skin resection and was placed under a fluorescent macroscope (microscope: MVX10, Olympus; light: PRIOR Lumen 1600-LED; camera: Flash 4.0 digital, Hamamatsu). The pump infusion (1 μL, 0.2 L/min) was triggered simultaneously with the imaging over the vein, once every 15 min for 2 h for the intraparenchymal delivery. PBS (1 μl) with or without 5 μM PGF_{2α} was applied directly onto the bilateral superficial cervical lymph vessels for the duration of the imaging. At the end of the experiment, animals were decapitated, and their brains were harvested for postmortem analysis. For quantitative image analysis for femoral vein and ex vivo in the brain is the same as described in Pla, V. et al. A real-time in vivo clearance assay for quantification of glymphatic efflux. Cell Rep 40, 111320 (2022).

### Statistical Information

All statistical analyses were performed on GraphPad Prism 8 and MATLAB. Data in all graphs are plotted as mean±standard error of the mean (SEM). Tests were chosen based on the data set and are reported in the figure legends. The analysis methods include 2-way ANOVA, Sidak's multiple comparisons test; one-way ANOVA, Tukey's multiple comparisons test; paired 2-tailed t test. P<0.05 was considered statistically significant. All bar graph data is presented as mean±standard error of the mean (SEM).

### Example 2 Quantifying Flow in Cervical Lymph Vessels

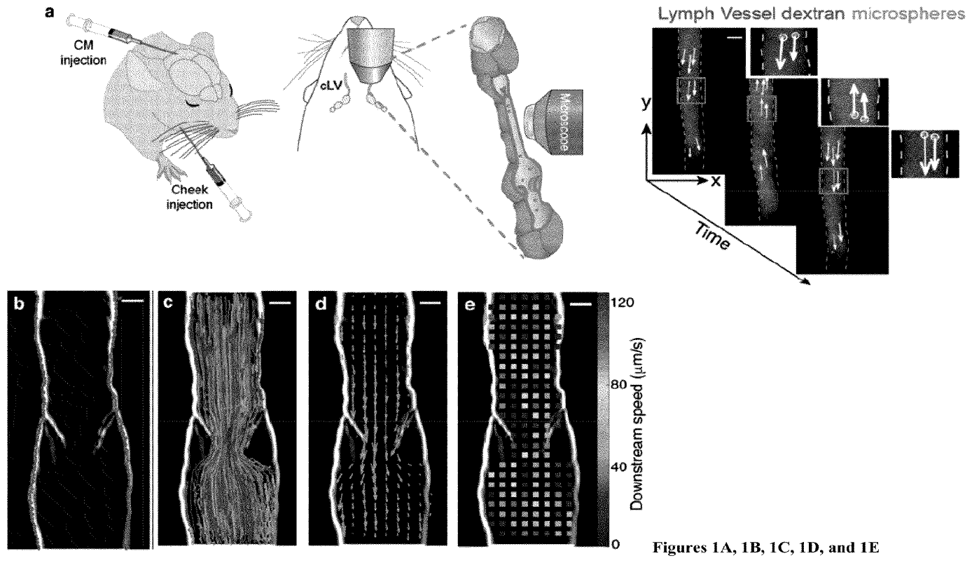

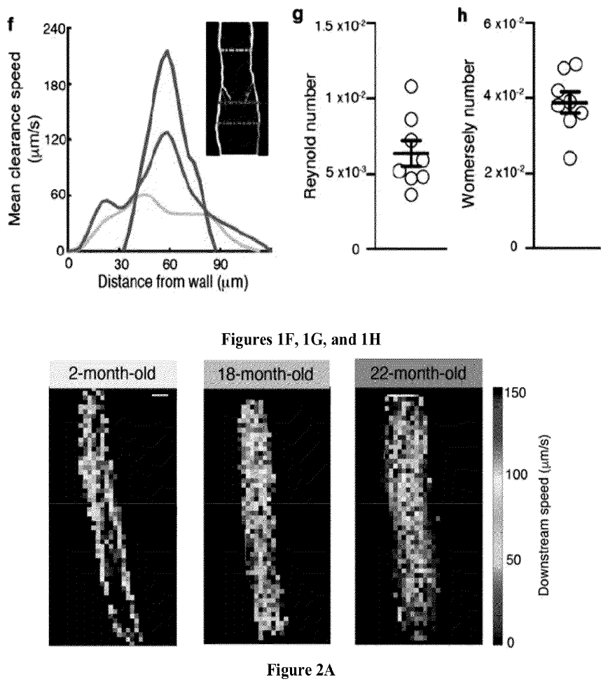

Cervical LVs are divided into superficial (scLV) and deep (dcLV) chains that together drain fluid from the CNS. However, these are also responsible for draining lymph from all remaining extracranial structures in the head and neck. To differentiate the transport properties of CNS-specific solutes, inventors used a tracer mapping approach which was termed 2P-OPTIC (FIG. 1a). To image cLVs and reduce photon scattering, both scLVs and dcLVs were surgically exposed. Superficial LVs lie just below the skin and can be seen through the superior cervical fascia, draining to superior cervical lymph nodes. Because dcLVs sit medial to the common carotid artery and the internal jugular vein, accessing them for visualization requires medial retraction of the trachea and medial musculature (sternohyoid and omohyoid muscle) and lateral retraction of the lateral musculature (sternomastoid and posterior belly of the digastric muscle).

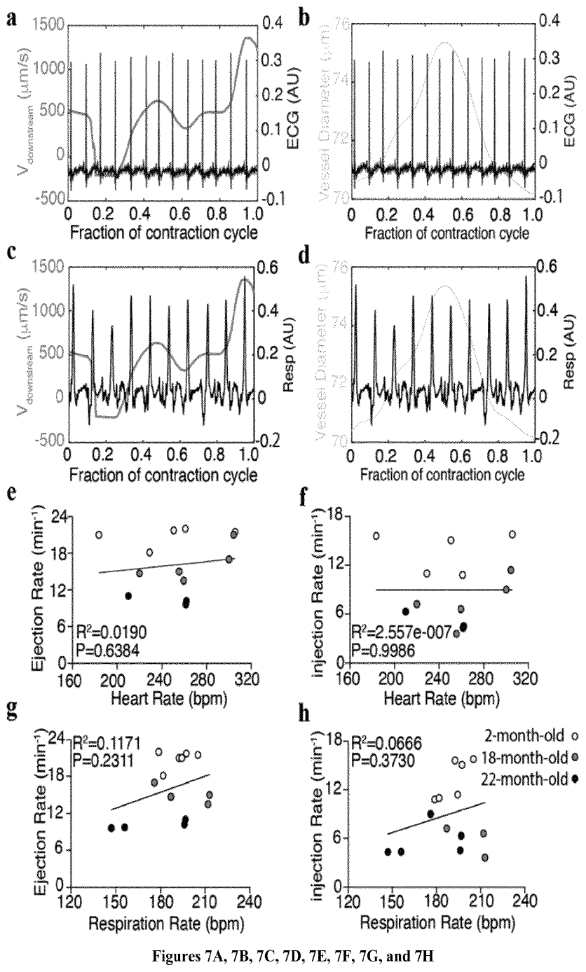

LVs were labelled by intramuscular cheek injection of FITC-dextran (3 kDa). Two-photon resonant scanning microscopy was used to acquire 60 Hz frame rate imaging of a single focal plane at a cLV. The dextran labelled the draining lymph and enabled the measurement of the internal diameter of the vessel (FIG. 1a). However, to parse out CNS-borne transport from extracranial drainage, 1 μm fluorescent spheres were injected into the CSF at the cisterna magna²⁰. Particle tracking velocimetry were then used to quantify flow speeds within the cLVs. The cardiac and respiratory cycle of the mouse were also recorded and therefore allowed simultaneous measurements of flow speed, contractility (FIG. 1b), and the cardiac and respiratory cycles (FIG. 7).

In each experiment, more than 1000 particles were tracked during a 10-minute recording (FIG. 1c). Pre-nodal cLVs in 2-month-old young adult mice were 72.4±5.5 μm in diameter. Particle tracks were distributed nearly uniformly, with an increased concentration near the centre of the vessel when the particles transit through the valves. Fluid flowed rapidly, downstream into the lymphangion, while the lymphatic valve below remained closed and the vessel filled—in some cases inducing the vessel to expand further. With the downstream valve closed, there was then retrograde fluid motion within the lymphangion causing particles to flow backwards closing the upstream valve. This was followed by contraction of the lymphangion which expelled the fluid downstream to the next lymphangion along the LV.

The distribution of spatial and temporal averages of the CSF flow speeds within the lymph vessel (FIG. 1e) showed an average velocity of 151.7±28.4 μm/s and was greatest near the vessel centre and least near the walls (FIG. 1f), as expected for pressure-driven flows in a channel.

In experiments where flow through the valves was visualized, the highest centerline speeds were measured at the valves (FIG. 1f), consistent with fluid accelerating through a smaller cross-sectional area to maintain a constant flow rate. The Reynolds (Re) number is a non-dimensional ratio between the inertial and viscous forces, Re>1 indicates that inertia dominates and Re<1 shows that viscous forces dominate. The Re number of cLVs was 0.0064±0.0008 (FIG. 1g), which supports that viscous forces dominate and fluid is propelled by the pressure-differences created by the contraction of the vessel wall.

Another way to characterize the flow is calculating a Womersley (Wo) number which describes the ratio of pulsatile flow frequency to viscous effects. The Wo number of cLVs 0.039±0.003 fell well below the Wo=0.1 limit (FIG. 1h), suggesting that transient inertial effects are negligible and fluid is primarily driven by lymphangion contractility. These represent the first descriptions of hydrodynamical properties of CSF drainage via cervical lymphatic vessels in live rodents.

Deep cervical LVs had similar flow characteristics compared to scLVs; however, due to their proximity to the carotid artery and the trachea their visualization was subject to significant motion artifacts. The frequency of the oscillatory artifacts was around ~3.18 Hz which coincides with the cardiac cycle frequency of the mouse, suggesting that cardiac pulsatility was the largest source of motion. Nonetheless, mean downstream flow speeds in dcLVs were comparable to those observed in scLVs (166.2±31.6 μm/s vs 151.7±28.4 μm/s; P=0.7423), with the flows in both lymphatic chains being laminar, viscosity-dominated flows (Re<<1). The total number of CNS-borne microspheres was also similar across the two LV chains (scLV: 129.6±28.0 particles/136 min vs. dcLV: 125.2±22.0 particles/min; P=0.9069), suggesting that both superficial and deep cLVs play a similar role in CSF drainage.

### Example 3 Lymphatic Fluid Drainage is Impaired in Aging

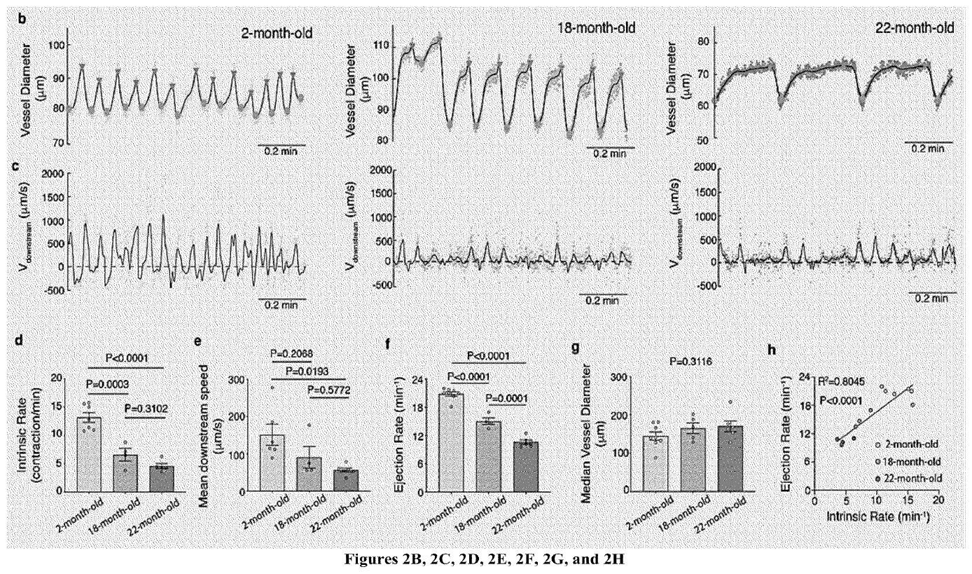

Efflux of CSF along cervical lymphatics declines in aged mice⁵, but whether this is due to delayed clearance from the intracranial compartment or due to an intrinsic effect of aging on LV function has not been described. Inventors evaluated the structure and function of cLVs using 2P-OPTIC in 2-, 18-, and 22-month-old mice (FIG. 2).

Re number in 18-(0.0052±0.0011) and 22-month-old (0.0041±0.0008, P=0.2194 vs 2 mo) and the Wo number (0.037±0.003 and 0.033±0.003 respectively, P=0.344) were comparable to those seen in young animals, indicating viscous dominant flows and negligible inertial forces. Vessel diameter was measured at 20 different spatial locations along the length of the vessel for over 10 minutes while simultaneously measuring the dynamics of lymphatic flow (FIG. 2b). There were no differences in median cLV diameter across ages, suggesting that cLVs reach adult size at 2-months of age (FIG. 2g). Despite similar sizes, average downstream velocity (V_{downstream}; FIGS. 2c and e) declined by ~40% at 18 months (90.9±29.1 μm/s, P=0.21 vs 2 mo) and was slowed significantly (~63%) by 22 months (56.6±5.7 μm/s, P=0.02 vs 2 mo). Cervical LVs expanded and contracted rapidly in young mice (13.2±0.8 contraction/min; FIG. 2d), with each contraction cycle lasting around 4.6 s (FIG. 2d). This is consistent with previous studies that measured contraction dynamics in collecting lymphatic vessels in the leg and flank of young mice under a ketamine-xylazine anaesthesia²¹. The intrinsic rate steeply declined in 18-month-old mice (50.0%, P=0.0003) and further declined in 22-month-old mice (65%, P<0.0001) (FIG. 2d), suggesting an age-dependent slowing of the contractility of cLVs.

In order to evaluate lymphatic function, assays were carried out to quantify the ejection rate or the frequency at which fluid was ejected from the lymphangion (FIG. 2f). This ejection rate does not necessarily match the intrinsic contraction rate but effectively represents the frequency with which the valves open and close and can be used to evaluate the efficiency of valve function. Inventors observed significant declines in the ejection rate from 2 to 18 months (−27.8%, P<0.0001) and from 18 to 22 months of age (−29.3%, P=0.0001) (FIG. 2f). These declines in ejection rate are strongly correlated to the impairment in the intrinsic contraction rate [coefficient of determination (R²)=0.8045; FIG. 2h] meaning that lymphangion contractility drives most of the flow. However, contrary to prior in vitro studies using isolated LVs, inventors did not observe a consistent one-to-one relationship between vessel contraction and flow oscillation^{17 22} suggesting that in vivo the upstream and downstream lymphangions influence flow within the lymphangion which alludes to the importance of synchronization between smooth muscle cells to drive coordinated flow along the vessel. Interestingly, lymphatic flow in cLVs was not dependent on heart or respiratory rate (FIG. 7). These experiments showed that in aging, cervical lymphatic vessels have significantly slowed contraction frequency which results in reduced lymphatic transport suggesting that the transport of solutes from the CNS to the cLN and into the periphery is slowed in aged animals.

### Example 4 Lymphatic Efflux Volumes are Reduced in Aging

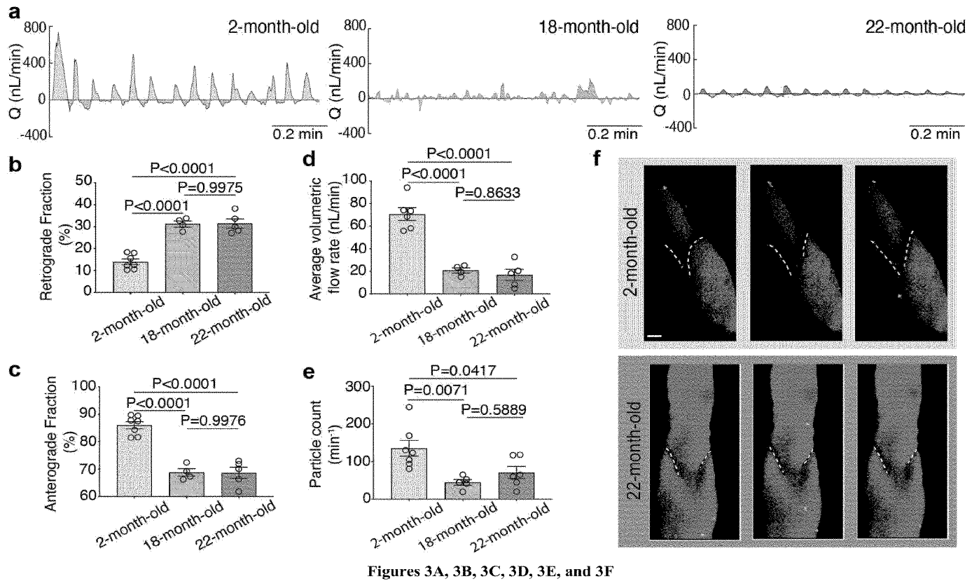

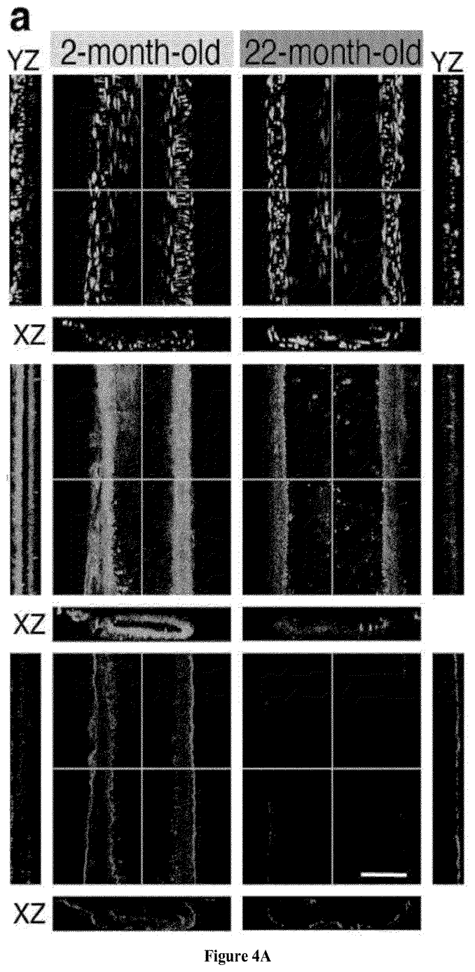

To assess instantaneous volumetric flow rate (Q), inventors calculated the product of the instantaneous downstream velocity and the instantaneous cross-sectional area (computed using the instantaneous median vessel diameter, assuming a circular cross section, FIG. 3a). Maximum flow rates in young mice reached 724.2 nL/min, while flow rates drastically decreased in aged mice (18-month: 423.2 nL/min; and 22-month: 180.9 nL/min).

Next, inventors computed the ejection fraction which is the percentage of the flow in the anterograde direction through the lymphangion and the retrograde fraction which corresponds to the percentage of flow in the opposite direction (FIGS. 3b-d). These two fractions are also known, respectively, as the anterograde fraction, defined as the fraction of the time during which fluid flows away from the skull, and the retrograde fraction, defined as the fraction of time during which flow is retrograde.

Not surprisingly, 2-month-old mice exhibited the highest ejection fractions expelling 85.9±1.3% of fluid downstream with only 13.9±1.3% of the flow moving retrogradely indicating that lymphatic flow in young mice are primarily unidirectional and are consistent with regulation by functioning unidirectional valves between lymphangions. Instead, aged mice exhibited a ~20% decrease in ejection fraction compared to young mice with 18- and 22-month-old mice pumping only 68.8±1.4% (P<0.0001 vs 2 mo) and 68.6±2.0% (P<0.0001 vs 2 mo) of fluid in the forward direction, respectively (FIG. 3c). The reduction in anterograde flow coincided with an impressive 2.2-fold increase in the retrograde flow in 18-(31.2±1.4%, P<0.0001 vs 2 mo) and 22-month-old mice (31.4±2.0%, P<0.0001 vs 2 mo) (FIG. 3b). Further inspection of the flows in old mice revealed that valves do not fully close (FIG. 3f) causing particles to oscillate back and forth without being entrained in a lymphangion. This suggests that aged valves are unable to limit reflux, resulting in the increased retrograde fractions and the decline in ejected fractions measured in aged mice.

Inventors functionally characterized the degree of impairment by computing the ejection volume, which is the volume of fluid effectively transported downstream the vessel per minute. In aged mice, less-frequent contractions combined with the higher retrograde fractions and lower ejection fractions, resulted in significantly lower volumes being ejected in 18-(20.8±2.3 nL/min, P<0.0001) and 22-month-old mice (16.9±4.9 nL/min, P<0.0001) compared to 2-month-old mice (70.6±5.6 nL/min, FIG. 3d). For comparison, this would equate to aged cLV transporting only about a quarter (~24.3 μl/day) of the lymph that is drained by young cLVs (~102 μl/day). It was hypothesized that this reduction in flow would also decrease the arrival of antigens and leukocytes to the cLN. To test this idea, inventors calculated the number of 1p m particles that are transported per minute as a surrogate of solute transport in the micron range (FIG. 3e). The analysis revealed that particle counts are also significantly decreased in 18-(44.5±7.5) and 22-month-old mice (62.4.5±16.1) compared to 2-month-old mice (104.8±19.1) (63.9% and 49.4%, respectively), indicating that lymphatic efflux is severely compromised in aging vessels.

### Example 5 Age-Induced Smooth Muscle Cell Loss Impairs Lymphatic Vessel Contractility

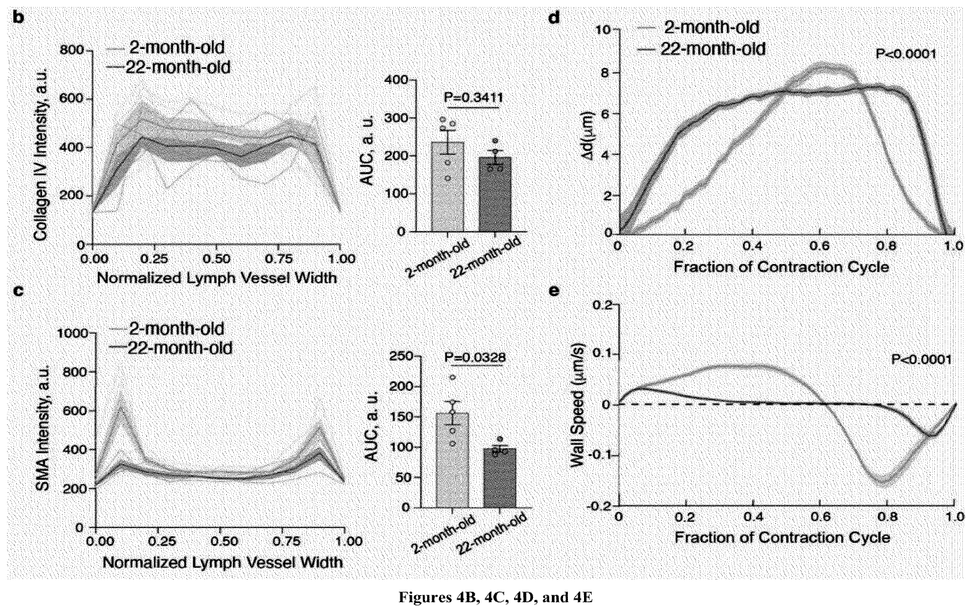

Aged collecting LVs display reduced contractility due to the loss of smooth muscle cell (SMC) layers lining the lymphangions, decreased expression of contractile proteins in SMCs, and changes in the composition of the basement membrane. Since aging is also associated with hyperplasia and dysmorphic in LVs¹¹, inventors aimed to determine whether an age-dependent change in the basement membrane properties could drive biomechanical differences that would explain the flow changes. Inventors performed immunohistochemical staining of cLVs labelling for collagen IV (Col IV), a main structural protein of the lymphatic basement membrane. Quantification of the basement membrane thickness and collagen IV expression showed no significant difference between young and aged animals (P=0.3411, FIGS. 4a-b), suggesting that the structural scaffolding of the cLV is retained across aging.

It was then asked if a loss of smooth muscle cell (SMC) layers, which are responsible for spontaneous, phasic contractions in LVs could contribute to the decline in contractility. SMCs were identified by staining for α-smooth muscle actin^{24 24}, the primary contractile protein in these cells. SMA fluorescence was 37.5% lower in 22-month-old mice compared to their young counterparts (P<0.0328), suggesting a significant loss in SMC investiture and contractile ability. Together, these results show that loss of SMCs via reduced expression of SMA could explain the observed decline in fluid transport seen in aged vessels. This decline in SMA expression also matches that seen in mesenteric lymph vessels in aging¹³, which might support the notion that there is a systemic loss of lymphatic SMCs.

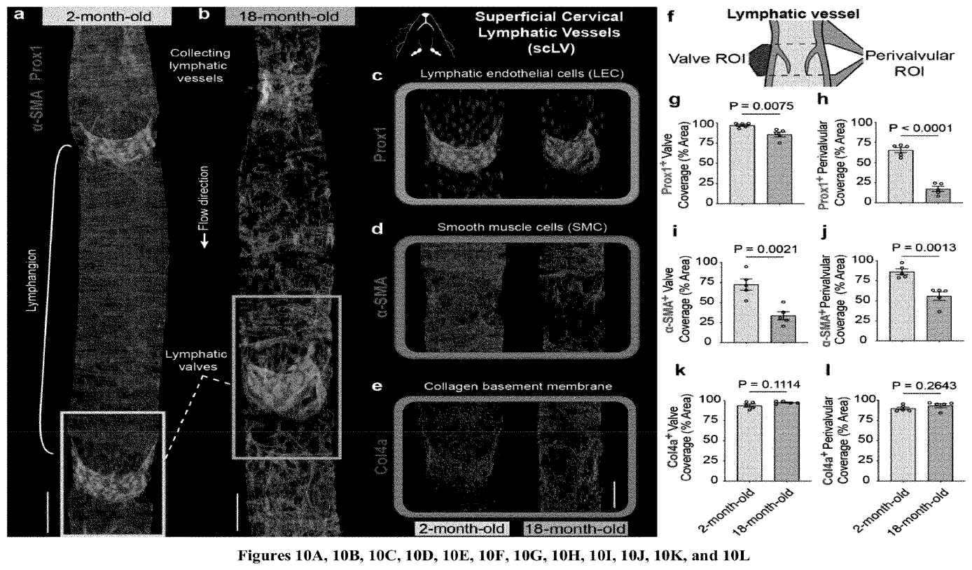

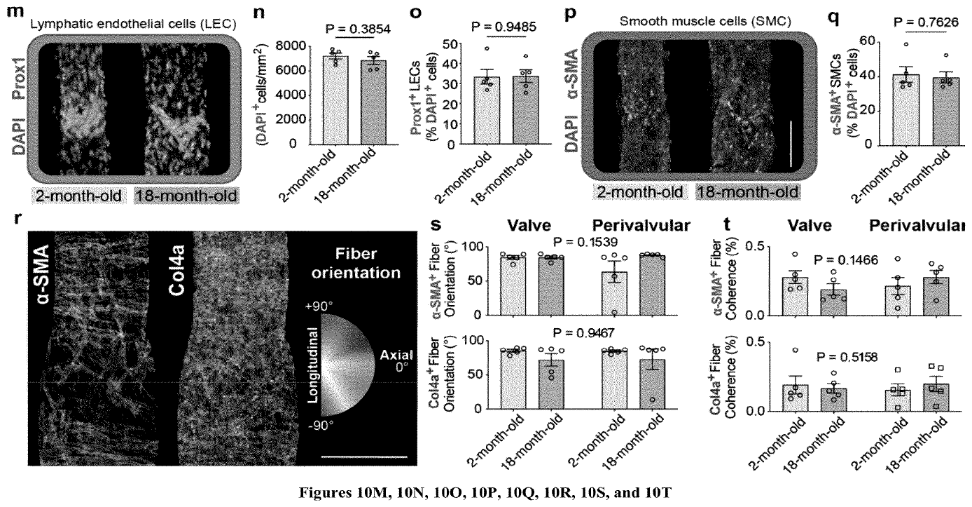

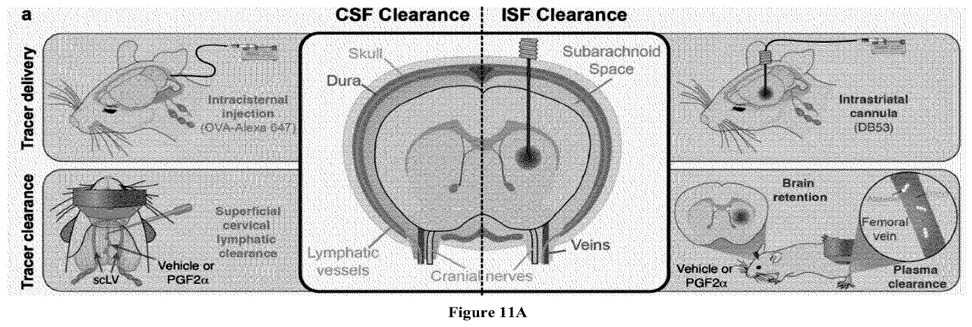

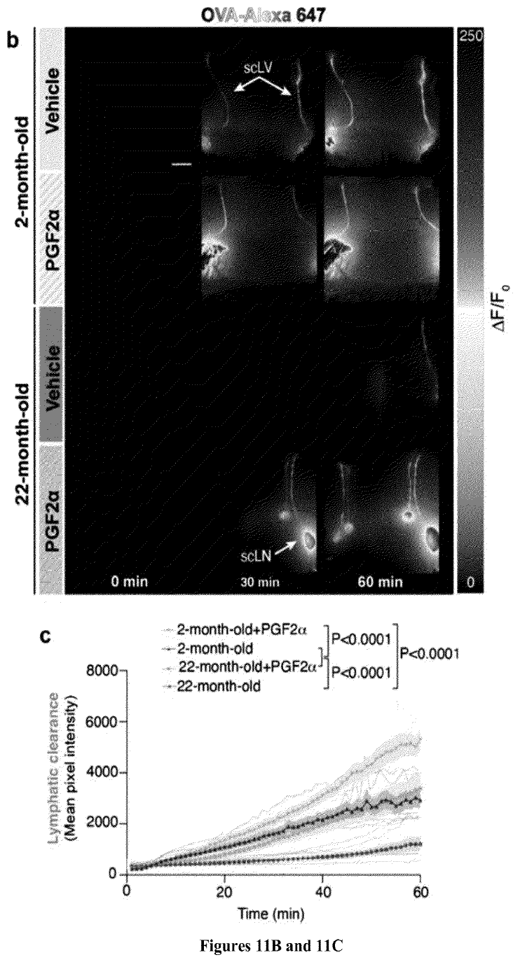

Additional immunohistochemical staining assays of cLVs in 2- and 18-month-old Prox1-GFP animals (FIG. 10a-b) were carried out to identify lymphatic endothelial cells (LECs, FIG. 10c), α-smooth muscle actin (αSMA, FIG. 10d) for SMCs, and collagen 4a (Col4a, FIG. 10e), a main structural protein of the lymphatic basement membrane. There was significant loss in the expression area of Prox1 and αSMA in both the lymphatic valve and the perivalvular regions of scLVs in aged mice. LECs (Prox1-positive area) covered around 12% less of the lymphatic valve in 18-month-old animals compared to young controls (97.2±1.3% vs. 85.8±2.9%; P=0.0075) and 74% less of the perivalvular portion of the LV (65.5±3.3 vs 17.1±3.1, P<0.0001). SMCs (αSMA-positive area) covered 54% less of the valve (72.6±7.1 vs 33.6±5.1, P=0.0021) and 35% less of the perivalvular region (86.2±3.5 vs 55.9±5.2, P=0.0013; FIGS. 10f-j). A similar relationship for SMC coverage was noted in 22-month-old animals. αSMA fluorescence was 37.5% lower in 22-month-old (97.5±5.6 arbitrary units, AU) mice compared to their young counterparts (156.1±19.0 AU, P=0.0328) (FIGS. 10a and c), suggesting a significant loss in αSMA and contractile ability. Interestingly, aging caused a 1.5-fold greater loss of SMC coverage at valves compared to perivalvular regions. Valvular αSMA is thought to actively control valve leaflet movement in other rodent lymphatic chains, and its loss could increase reflux and decrease lymphatic efflux, as we observed in aged mice.

Quantification of the basement membrane thickness and Col4a expression showed no significant difference between 2-month-old and either 18- and 22-month-old animals (FIGS. 10k-l and FIGS. 4a-b), suggesting that the structural scaffolding of the cLV is retained across aging.

It was then asked if the decreased expression of Prox1 and αSMA was the result of either global cell loss or rather a cell-specific process. LECs are responsible for channeling flow, and SMCs drive spontaneous, phasic contractions in LVs. A reduction in either of these could decrease contractility. LECs were identified as DAPI+/Prox1+ cells while SMCs were identified as being DAPI+/αSMA+39. Results showed that total cell, LEC, and SMC numbers are comparable between 2-month-old and 18-month-old animals (total cell: P=0.3854; LEC: P=0.9485; SMA: P=0.7626) (FIGS. 10m-q).

Assays were carried out to examine if abnormal fibre orientation of both αSMA and Col4a fibres could contribute to the anomalous contractility noted in aged animals (FIG. 10r). However, it was found that fibre orientation and orientation coherence for both markers were preserved in the valvular and perivalvular regions in aging (orientation: P=0.1539; coherence: P=0.1466) (FIGS. 10s and t).

Together, these results show that reduced coverage of αSMA and Prox1, rather than fewer LECs or SMCs, could explain the observed decline in fluid transport seen in aged vessels. This decline in SMA expression also matches that seen in mesenteric lymph vessels in aging, which might support the notion that this is a systemic process rather than a regionally specific event.

To further characterize the cLV wall dynamics, inventors computed phase-averaged vessel diameter waveforms and calculated the average contraction amplitude over the systolic cycle (FIG. 4d). Inventors observed a 20% reduction in the contraction amplitude and a 30% longer contraction phase of 22-month-old vessels, compared to 2-month-old vessels (P<0.0001, FIG. 4d). Two-month-old cLVs achieved wall velocities double that of their aged counterparts, indicating faster and more efficient pumping (P<0.0001, FIG. 4e).

In summary, these results suggest that reduced SMA fibre coverage or SMC loss, rather than basement membrane changes, coincides with slower and smaller intrinsic pulsations, which together with decrease contraction frequency (FIG. 2d) result in the lower average volumetric flow rates (FIG. 3d) in aging.

### Example 6 PGF_{2α} Restores cLVs Pumping by Improving Lymph Vessel Contractility

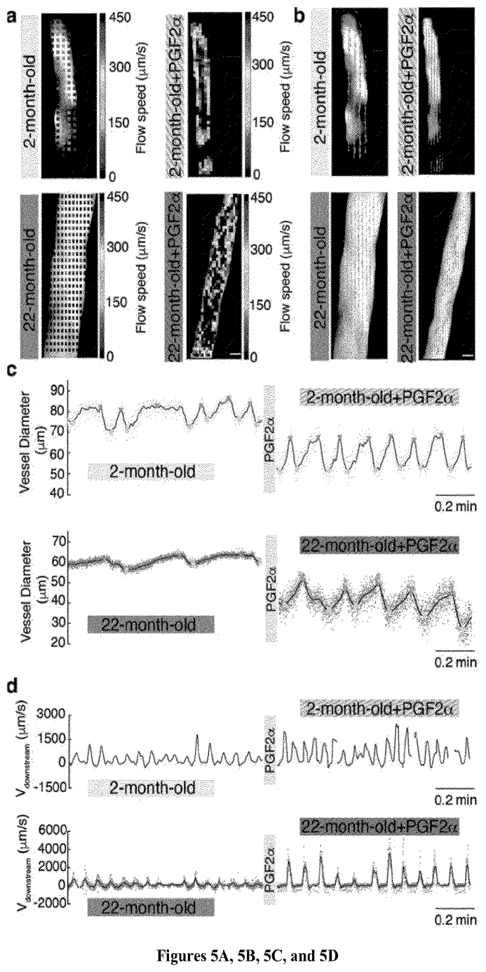

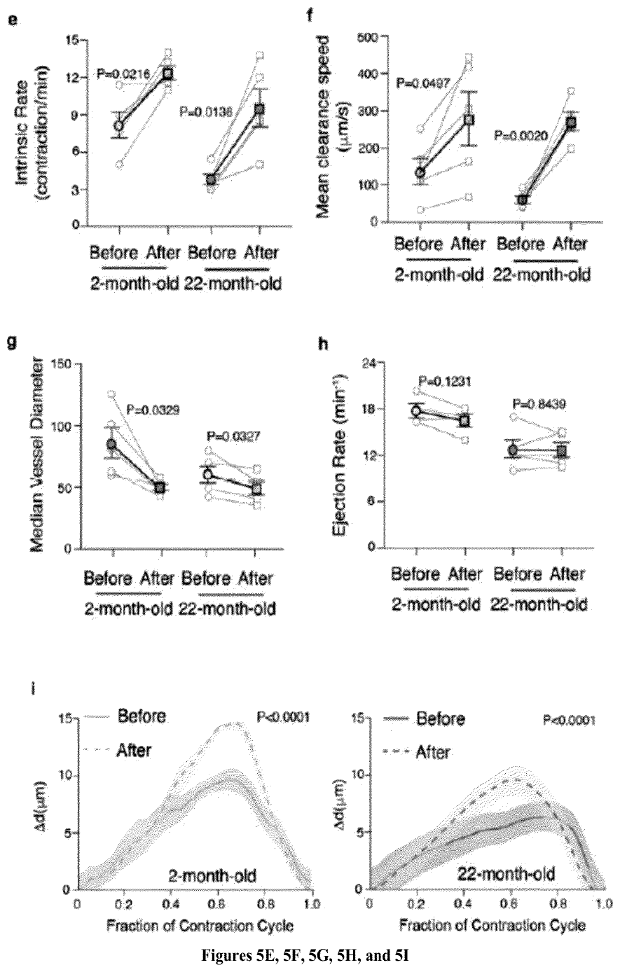

Since intrinsic pulsations are the principal driver of cLV transport, and both LEC and SMC numbers are preserved in aged cLVs, it was asked if rescuing the function of remaining SMCs in aged cLVs might restore lymphatic drainage. In order to test this, prostaglandin F_{2α} (PGF_{2α}) was topically applied (FIG. 5a-d). PGF_{2α} is a prostanoid that has been shown to induce LVs contractions by increasing intracellular Ca²⁺ concentrations in SMC^{19,26,27},

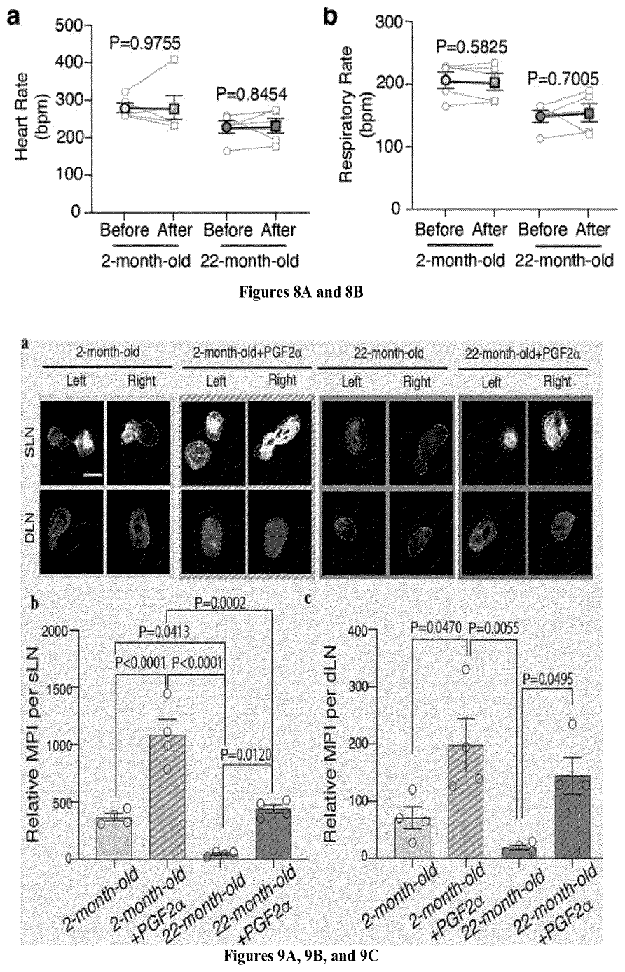

It was found that PGF_{2α} dramatically increased the intrinsic pulsation rate (FIG. 5e) not only in young mice (50.7%, P=0.0216), but even more so in old mice (152.0%, P=0.0136), rescuing the intrinsic rate back to levels seen in young animals prior to treatment (P=0.487). The increased pulsation rate also resulted in faster downstream speeds in young mice (103.1%, P=0.0497); however, in old mice PGF_{2α} exposure triggered a 3.5-fold increase in flow speeds (P=0.0020), rescuing flow and even surpassing baseline speeds in young animals (P=0.016, FIG. 5f). Notably, PGF_{2α} decreased the median vessel diameter in 2-(P=0.0329) and 22-month-old mice (P=0.0327, FIG. 5g), but also increased the contraction amplitude (Δd, P<0.0001 vs before, FIG. 5i), suggesting that in addition to increasing phasic activity it also enhances tonic activity. However, there was no change in the ejection rate in 2- (P=0.1220) or 22-month-old mice (P=0.8362, FIG. 5h), indicating that PGF_{2α} primarily stimulates SMC contractions but has no effect on valve dynamics. Reassuringly, topical application of PGF_{2α} did not induce changes in the cardiac or respiratory rates (FIG. 8). These experiments confirmed that PGF_{2α} rescued the intrinsic pumping ability of the aged vessels by amplifying the contraction amplitude (leading to increased volumetric changes), and by boosting contraction frequency (fostering the faster ejection of fluid). These observations further support the idea that age-induced cLV dysfunction can be reversed by optimizing SMC function.

To quantify whether boosting vessel function with PGF_{2α} directly translated to improved cLV transport, inventors calculated volumetric flow rates and characterized the ejection and retrograde fractions after treatment (FIG. 6). It was observed that after PGF_{2α}, lymphatic flow was predominantly unidirectional, with little retrograde flow (FIG. 6a,). Ejection fraction was increased by 7.0% in 2-month-old mice (90.8±0.8 vs 97.1±0.6, P=0.0045) and by 31.7% in 22-month-old mice (P=0.0005, 72.5±2.1 vs 95.5±0.5, FIG. 6c), while the retrograde fraction was decreased by 64.2% in 2-month-old mice (8.1±1.0 vs 2.9±0.6, P=0.0061) and by 83.6% in 22-month-old mice after PGF_{2α} (27.5±2.1 vs 4.5±0.4, P=0.0005, FIG. 6b). The decreased retrograde flow resulted in significantly larger ejection volumes after PGF_{2α} in both 2-month-old mice (148.6%, P=0.0021) and 22-month-old mice (181.4%, P=0.0215) (FIG. 6d). More importantly, treatment with PGF_{2α} was able to overcome aged-induced lymphostasis and increased the ejection fraction and reduced the retrograde fraction even when compared with untreated young controls (P=0.0012 and P=0.0133, respectively). PGF_{2α} also rescued the lower ejection volumes in aged animals (P=0.0017), implying that with adequate therapy, the function of aging lymphatic vessels can be restored to youthful levels.

### Example 7 Restoring Cervical LV Function Rescues Impaired CSF Drainage in Aged Animals

Since cLVs are responsible for draining CSF from the CNS, it was asked whether restoring cLV function with topical administration of PGF_{2α} would enhance CSF clearance.

After intracisternal delivery of fluorescent microspheres, the number of particles detected in the cLVs was quantified as a surrogate of solute transport. CSF drainage in 2-month-old mice increased 35.4% after administration of PGF_{2α} (P=0.0355, FIG. 6e). Surprisingly, in aged mice treatment resulted in nearly 4 times more solute arriving to the cLN compared to untreated aged controls (P=0.0017). Cervical LV stimulation alone was sufficient to enhance CSF drainage in aged mice above young untreated levels (P=0.0208). Moreover, since cLVs transport fluid via bulk motion, increased CSF clearance should apply to all macromolecular solute immersed in the fluid (e.g., proteins and cells). To test whether PGF_{2α} could increase the efflux of proteins in the CSF, ovalbumin conjugated to Alexa Fluor 647 (OVA647) was injected into the cisterna magna. Since 2P-OPTIC is only able to quantify transport along a single cLV, to include all superficial cLVs an epifluorescent macroscope was used to quantify net transport of a protein-sized tracer (45 kDa) from the CSF. PGF_{2α} treatment was also extended to 60 min to identify if its effect could be sustained for longer durations. CSF clearance was inferred from changing fluorescence intensity of the OVA647 tracer after cisterna magna injection (FIG. 6f).

It was found that PGF_{2α} promoted robust CSF drainage in both young (P<0.0001) and old mice (P<0.0001, FIG. 6f), and rescued the blunted CSF efflux seen in 22-month-old animals back to 2-month-old levels. Ex vivo analysis of the cLNs showed a significantly larger accumulation of OVA647 after PGF_{2α} in 2- and 22-month-old mice (FIG. 9).

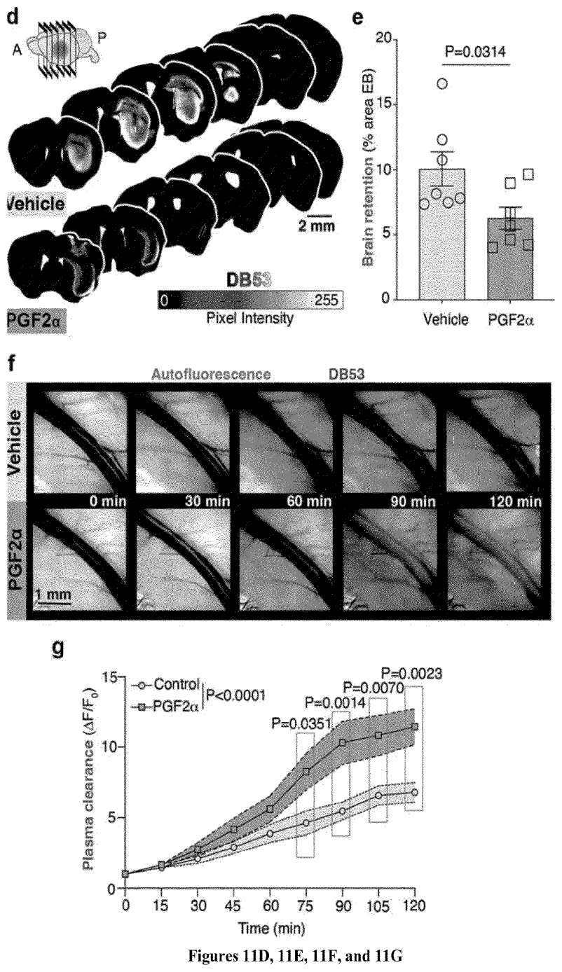

Drainage of a protein tracer delivered into the cisterna magna can bypass the brain parenchyma, so to further evaluate the role of augmenting cLV transport on brain clearance, inventors labelled interstitial fluid (ISF) in the striatum by injecting Direct Blue 53 (DB53, also T-1824 or Evans Blue) (FIG. 11a). DB53 was chosen because it is a small (960.8 Da), fluorescent tracer that reliably traces ISF. DB53 is cleared towards blood, where it has high affinity to plasma albumin, allowing it to remain in circulation for up to 135 min at stable concentrations. The degree of DB53 that is ultimately retained in the brain was significantly decreased after PGF_{2α} (6.3±0.9 vs 10.1±1.3; P=0.0314) (FIGS. 11d-e). Accordingly, brain clearance can be dynamically quantified by assessing DB53 signal in the femoral vein, which was dramatically increased after PGF2a compared with control (P<0.0001) (FIGS. 11f-g). The DB53 signal was significantly higher with PGF2a compared with control at 75 min (8.3±1.3 vs 4.6±0.8; P=0.0351), 90 min (10.3±1.5 vs 5.5±0.6; P=0.0014), 105 min (10.8±1.4 vs 6.6±0.7; P=0.0070) and 120 min (11.45±8.3 vs 6.8±0.7; P=0.0023) (FIG. 11g) In summary, restoring cLV function boosts CSF drainage and increases the arrival of CSF-borne proteins to cLNs in both young and aged mice. It also suggests that cervical lymphatics are important drivers of CSF clearance and function as critical bottlenecks in slowing CSF outflow in old age.

The foregoing examples and description of the preferred embodiments should be taken as illustrating, rather than as limiting the present disclosure as defined by the claims. As will be readily appreciated, numerous variations and combinations of the features set forth above can be utilized without departing from the present disclosure as set forth in the claims. Such variations are not regarded as a departure from the scope of the disclosure, and all such variations are intended to be included within the scope of the following claims. All references cited herein are incorporated by reference in their entireties.

1. A method for improving glymphatic-lymphatic efflux from the central nervous system (CNS) of a subject, comprising enhancing the flow rate of a cervical lymphatic vessel (cLV) of the subject.

2. The method of claim 1, wherein said enhancing the flow rate comprises increasing the contraction of the cLV.

3. The method of claim 2, wherein said increasing the contraction of the cLV comprises administering to the subject an agonist of smooth muscle contraction.

4. The method of claim 3, wherein the agonist comprises a muscarinic agonist, an α1-adrenergic agonist, a K_{ATP} channel inhibitor, a NOS inhibitor, a, soluble guanylate cyclase (sGC) blocker, a PKA inhibitor, a cyclooxygenase-1 (COX-1) inhibitor, a cyclooxygenase-2 (COX-2) inhibitor, a prostanoid, a prostaglandin E2 receptor 4 (EP4 receptor) antagonist, a prostacyclin (PGI2) receptor (IP receptor) antagonist, or a nonsteroidal anti-inflammatory drug (NSAID).

5. The method of claim 4, wherein the agonist is selected from the group consisting of carbachol, norepinephrine, propranolol, glibenclamide, NG-nitro-L-arginine (L-NNA), ODQ, H-89, KT5720, indomethacin, ketoprofen, AH23848B, GW627368x, CAY10441, pyridostigmine, amifampridine, neostigmine, edrophonium, and ambenonium.

6. The method of claim 4, wherein the agonist comprises a lymphatic contractility-promoting prostanoid.

7. The method of claim 6, wherein the prostanoid comprises prostaglandin F_{2α} (PGF_{2α}), thromboxane A₂ (TxA₂), PGD₂, leukotriene B4, leukotriene C4, leukotriene D4, or TxA₂ mimetic U46619.

8. A method for improving glymphatic-lymphatic efflux from the CNS of a subject, comprising administering to the subject an inhibitor of phosphodiesterase type 5a.

9. The method of claim 3, wherein the agonist or inhibitor is administered to the neck of the subject.

10. The method of claim 9, wherein the agonist or inhibitor is administered topically.

11. The method of claim 1, further comprising one or more of the following,
increasing influx of cerebrospinal fluid (CSF) through the CNS interstitium, and
messaging the neck of the subject.

12. The method of claim 11, wherein the increasing influx of CSF comprises administering to the subject an agent selected from the group consisting of a hypertonic solution a Stat-3 inhibitor, a bone morphogenetic protein (BMP) signaling axis molecule, an antagonist of AVP (vasopressin), an antagonist of atrial natriuretic peptide (ANP), an antagonist of Angiotensin II, an antagonist of AT2R receptors, and an antagonist of AT1 receptors.

13. The method of claim 12, wherein the hypertonic solution comprises NaCl or mannitol.

14. A method for promoting clearance of a substance from the CNS interstitium, brain interstitium and/or spinal cord interstitium of a subject, comprising improving glymphatic-lymphatic efflux from the CNS of the subject according to claim 1.

15. The method of claim 14 wherein the substance comprises a fluid or a solute.

16. The method of claim 14 wherein the substance comprises amyloid β (Aβ), tau, or alpha synuclein or comprises a drug or a metabolite thereof.

17. (canceled)

18. A method for treating a cerebral edema, a traumatic brain injury, a post-traumatic brain injury, or a neurodegenerative disease in a subject thereof, comprising improving glymphatic-lymphatic efflux from the CNS of the subject according to claim 1.

19. A method for treating a cerebral edema, a traumatic brain injury, a post-traumatic brain injury, or a neurodegenerative disease in a subject in need thereof, comprising administering to the subject an inhibitor of phosphodiesterase type 5a.

20. The method of claim 8, wherein the inhibitor is selected from the group consisting of sildenafil, tadalafil, vardenafil, and avanafil.

21. The method of claim 18, wherein the neurodegenerative disease is Parkinson's disease (PD), Alzheimer's disease (AD), Alzheimer's disease with Lewy bodies, Lewy body dementia, mixed dementia, vascular dementia, frontotemporal dementia, chronic traumatic encephalopathy (CTE), HIV associated dementia Lewy body disease, Huntington disease, or multisystem atrophy.

22-25. (canceled)
